(19) United States
(12) Patent Application Publication
(10) Pub. No.: US 2025/0009293 A1
(43) Pub. Date: Jan. 9, 2025

(54) PLURIPOTENT SMART MEDICAL DEVICES, MICROBOTS AND NANOBOTS

(71) Applicant: Bruce REINER, Berlin, MD (US)

(72) Inventor: Bruce REINER, Berlin, MD (US)

(21) Appl. No.: 18/759,049

(22) Filed: Jun. 28, 2024

Related U.S. Application Data
(60) Provisional application No. 63/511,990, filed on Jul. 5, 2023.

(51) Int. Cl. A61B 5/00; A61B 10/04; A61B 34/00; A61B 34/30; A61B 90/00; A61B 90/90; B82Y 5/00; G16H 40/67

(52) U.S. Cl. CPC A61B 5/4836; A61B 10/04; A61B 34/30; A61B 34/72; A61B 90/90; G16H 40/67; A61B 2034/303; A61B 2090/365; A61B 2090/3735; A61B 2090/3782; A61B 2090/3966; A61B 2090/3983; A61B 2562/0285; B82Y 5/00

(57) ABSTRACT

The present invention relates to a medical device which monitors biological data, includes: a microscopic medical device configured to be disposed within a human body, the microscopic medical device having a plurality of components which operate in real-time to continuously obtain data from within the human body; a processor configured to receive the data from the plurality of components and analyze the data; wherein at least one of said plurality of components is utilized to change a structure, a form, or a content of the microscopic medical device based upon the analysis of the data; and wherein the microscopic medical device implements a medical intervention within the human body based upon said analysis of the data and the change in structure, form or content of the microscopic medical device.

### CROSS-REFERENCE TO RELATED APPLICATIONS

The present invention claims priority from U.S. Provisional Application No. 63/511,990, filed Jul. 5, 2023, the contents of which are herein incorporated by reference in its entirety. The present application is related to U.S. Pat. No. 11,324,451, U.S. patent application Ser. No. 17/575,048, filed Jan. 13, 2022, and Ser. No. 17/712,693, filed Apr. 4, 2022; U.S. Pat. No. 11,224,382, and U.S. patent application Ser. No. 17/836,742, filed Jun. 9, 2022 (“the incorporated patents/applications”) the contents of all of which are herein incorporated by reference in their entirety.

### BACKGROUND OF THE INVENTION

### 1. Field of the Invention

The present invention relates to in vivo smart devices which can be fundamentally transformed in form and functionality, in response to the constantly changing host medical requirements. These structural and functional changes in the smart devices and components of the present invention can be derived in a number of ways, including through modification in software and/or hardware.

### 2. Description of the Related Art

In current practice, medical devices are constructed to exist in their final and all-inclusive form with little to no change in functionality. Whether one is considering a cardiac pacemaker, vascular catheter, biliary stent, or implantable pump; once these devices are positioned, they remain relatively stagnant and fixed in position, form and function. In the event that a given device was to become damaged, repositioned, or non-functional, it is inevitably destined for removal and/or replacement by an entirely new device. In many situations, the removal of these devices requires physical intervention in some form by a third party, which may take the form of an open surgery, endoscopy, or interventional radiology.

This belies the fact that present day medical devices are largely passive in nature and do not possess the intrinsic capabilities of self-repair, autonomous navigation, structural modification, or functional change.

In addition to these macroscopic medical devices which have largely defined human biotechnology to date, an entirely new call of microscopic devices in the forms of microbots and nanobots are in their developmental infancy. As biotechnology continues to advance and miniaturize, these will at some point in time become integrated in the arsenal of medical diagnosis and therapeutics. These offer a number of theoretical advantages to their macroscopic counterparts, based on their markedly reduced size and ability to reach anatomic locations which are not practical or feasible to achieve with conventionally sized medical devices.

Regardless of the specific class of medical devices being considered, there has been no previously known plans to dynamically reconstruct and/or redesign in vivo medical devices (irrespective of their size) into devices which are capable of transforming themselves in size, structure, form, content, or functionality.

Thus, a detailed and versatile plan to accomplish the above goals, with the ultimate objective of creating smart in vivo medical devices which can effectively become pluripotent, in an analogous manner to biologic stem cells, was needed.

### SUMMARY OF THE INVENTION

The present invention relates to in vivo smart devices which can be fundamentally transformed in form and functionality, in response to the constantly changing host medical requirements. The present invention is a novel utilization of the concepts of pluripotency and multipotency applied to biotechnology in the form of in vivo smart devices. More specifically, the present invention relates to in vivo smart devices which can be fundamentally transformed in form and functionality, in response to the constantly changing host medical requirements. These structural and functional changes in the smart devices and components of the present invention can be derived in a number of ways, including through modification in software and/or hardware.

The smart devices of the present invention can be designed in a variety of sizes, forms, and functionality and will heretofore be collectively referred to as “in vivo smart medical devices” (which encompass macroscopic smart medical devices, smart microbots, and smart nanobots). In addition to the smart devices themselves, each individual smart medical device can also contain a variable number of smart components, each of which has the ability to function independently.

In the present invention, in vivo smart medical devices possess the capability of modifying their form, content, structure, and functionality to essentially transform themselves into uniquely different smart bots with a shared lineage from their predecessor. This process of device differentiation can take a variety of forms including (but not limited to) changes in size, structure, format, components, transportability, or functionality. The net effect is the creation of a functionally and/or structurally new in vivo smart medical device which has unique capabilities and actions, which are distinct and separate from the smart medical device and/or smart components from which it originated.

An added benefit to the present invention is the ability to effectively expand the lifetime of a given smart medical device through the integration and/or replacement of its device subcomponents, which have a limited life expectancy commensurate with its mechanical attributes. While not the equivalent of biological self-renewal, it does offer the potential to redefine the existing challenges inherent to technological obsolescence. The net effect is that the existing paradigm of structurally and functionally fixed (or static) biotechnology can be replaced by a technology which is dynamic, responsive, and evolving in nature.

The foundation of the present invention is in part predicated upon a variety of methodologies generally described in related patents/applications by the present inventor including U.S. Pat. No. 11,324,451, U.S. patent application Ser. No. 17/575,048 filed Jan. 13, 2022, and Ser. No. 17/712,693 filed Apr. 4, 2022; U.S. Pat. No. 11,224,382, and U.S. patent application Ser. No. 17/836,742 filed Jun. 9, 2022 (“the incorporated patents/applications”) the contents of all of which are herein incorporated by reference in their entirety.

The technologies derived from the present invention will ultimately lead to an entirely new class of smart in vivo medical devices with the capabilities of real-time and dynamic transformation within the ever changing and complex milieu in which they reside. The ultimate goal is to transform medical diagnosis and treatment from a static to dynamic process, while creating a unique symbiosis with the intrinsic host's native biologic system.

In one embodiment, a medical device which monitors biological data, includes: a microscopic medical device configured to be disposed within a human body, the microscopic medical device having a plurality of components which operate in real-time to continuously obtain data from within the human body; a processor configured to receive the data from the plurality of components and analyze the data; wherein at least one of said plurality of components is utilized to change a structure, a form, or a content of the microscopic medical device based upon the analysis of the data; and wherein the microscopic medical device implements a medical intervention within the human body based upon said analysis of the data and the change in structure, form or content of the microscopic medical device.

In one embodiment, the change in structure, form or content to the microscopic medical device includes an addition, a removal or a modification of one of the plurality of components.

In one embodiment, the microscopic medical device has an articulated structure which allows at least one individual segment of the microscopic medical device to be removed and the microscopic medical device one of deconstructed into separate functioning microscopic medical devices or the individual segment of the microscopic medical device is replaced with another segment introduced externally into the articulated structure.

In one embodiment, the microscopic medical device is a biosphere and the plurality of components are a plurality of active, inactive or dormant biosensors which are activated, deactivated, or moved to a different position on the biosphere, to accomplish predetermined activities.

In one embodiment, the microscopic medical device includes one of a microbot or a nanobot, a catheter, a stent, a cardiac pacemaker, a biosphere, an inferior vena cava filter, or a storage device depot.

In one embodiment, at least one component of the plurality of components to effect the change in structure, form or content of the microbot or nanobot is introduced from a storage compartment of another microbot or nanobot or from an external storage depot which contains a plurality of components or microscopic medical devices.

In one embodiment, a first microbot or nanobot moves to a second microbot or nanobot using its own navigational system, or vice versa, or a third microbot or nanobot delivers the at least one component of the plurality of components from one to the other microbot or nanobot.

In one embodiment, the change in structure, form or content to the microscopic medical device changes function of the microscopic medical device and is reversible.

In one embodiment, the change in structure, form or content to the microscopic medical device changes function of the microscopic medical device and is reversible.

In one embodiment, each individual segment has its own navigation system.

In one embodiment, removal of one of the individual segments or a component of the plurality of components is for repair of the microscopic medical device.

In one embodiment, the processor runs a program that utilizes artificial intelligence (AI).

In one embodiment, the plurality of components and/or a plurality of microscopic medical devices are constructed or aggregated into a microscopic medical device with different functionality from the individual components and/or devices.

In one embodiment, the microscopic medical device is transformed and/or modified using software and/or hardware components.

In one embodiment, the processor is remotely activated or deactivated depending upon computer system security requirements.

In one embodiment, the plurality of the microscopic medical devices have autonomous function and intercommunication to accomplish the medical intervention.

In one embodiment, a system which monitors biological data, includes: a medical device which monitors biological data, includes: a microscopic medical device configured to be disposed within a human body, the microscopic medical device having a plurality of components which operate in real-time to continuously obtain data from within the human body; a processor configured to receive the data from the plurality of components and analyze the data; wherein at least one of said plurality of components is utilized to change a structure, a form, or a content of the microscopic medical device based upon the analysis of the data; and wherein the microscopic medical device implements a medical intervention within the human body based upon said analysis of the data and the change in structure, form or content of the microscopic medical device; and a device depot, which stores one of a plurality of components, a plurality of microscopic or non-microscopic medical devices, or pharmacologic medications, in individual compartments.

In one embodiment, the system further includes a plurality of navigational aids which assist the plurality of components or the plurality of microscopic medical devices to navigate and enter the device depot.

In one embodiment, the device depot is internal or external to the human body and the plurality of components or the plurality of microscopic medical devices are injected into the human body from the device depot.

In one embodiment, a method of monitoring biological data, includes: disposing a microscopic medical device within a human body, the microscopic medical device having a plurality of components which operate in real-time to continuously obtain data from within the human body; receiving data from the plurality of components using a processor, and analyzing the data; wherein at least one of said plurality of components is utilized to change a structure, a form, or a content of the microscopic medical device based upon the analysis of the data; and wherein the microscopic medical device implements a medical intervention within the human body based upon the analysis of the data and the change in structure, form or content of the microscopic medical device.

Thus, has been outlined, some features consistent with the present invention in order that the detailed description thereof that follows may be better understood, and in order that the present contribution to the art may be better appreciated. There are, of course, additional features consistent with the present invention that will be described below, and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

The description of the drawings includes exemplary embodiments of the disclosure and are not to be considered as limiting in scope.

FIG. 1A shows a smart device such as a smart biosphere having an inner layer and outer layer of biosensors, according to one embodiment consistent with the present invention.

FIG. 1B shows the smart biosphere of FIG. 1A, with dormant biosensors being exchanged with inactive biosensors, according to one embodiment consistent with the present invention.

FIG. 1C shows the smart biosphere of FIG. 1B after the dormant biosensors are moved to the outer layer and prior to activation, according to one embodiment consistent with the present invention.

FIG. 2 shows a plurality of internal host smart devices, including a smart biosphere, a smart medical device containing a storage compartment, and a smart device with sensors, communicating with an external computer system and/or an external smart device (i.e., smart watch) (both external devices which can communicate between themselves), according to a plurality of embodiments consistent with the present invention.

FIG. 3 shows a smart vascular catheter with a plurality of subcomponents/sensors, that can be segmented, with a removable segment being modified to remove particular subcomponents to end up with a segment that includes unique structure, content, and functionality, according to one embodiment consistent with the present invention.

FIG. 4 shows a plurality of smart devices and components specific to the treatment of thrombo-embolic disease, according to one embodiment consistent with the present invention.

FIG. 5 shows an ex vivo storage device such as an auto injector, showing only an internal view of the storage compartments, with the injection portion omitted, according to one embodiment consistent with the present invention.

FIG. 6A shows a smart catheter with a plurality of biosensors thereon, according to one embodiment consistent with the present invention.

FIG. 6B shows a smart catheter with two auxiliary components attached thereto, where one of the auxiliary components is detached to form a different functional device in addition to the smart catheter, according to one embodiment consistent with the present invention.

### DESCRIPTION OF THE INVENTION

The present invention relates to in vivo smart devices which can be fundamentally transformed in form and functionality, in response to the constantly changing host medical requirements. The present invention is a novel utilization of the concepts of pluripotency and multipotency applied to biotechnology in the form of in vivo smart devices. More specifically, the present invention relates to in vivo smart devices which can be fundamentally transformed in form and functionality, in response to the constantly changing host medical requirements. These structural and functional changes in the smart devices and components of the present invention can be derived in a number of ways, including through modification in software and/or hardware.

### A. Differentiation and Adaptability of Smart Medical Devices

The ability to transform the form and/or functionality of a smart device is in part determined by the physical size and footprint of the smart device. Smart medical devices with larger sizes offer the greatest amount of surface area to accommodate miniaturized devices and components, which to some extent will impact the ability of a given smart device to alter its functional abilities. (Note: throughout the specification, nanobots, microbots, smart devices, etc. are referred to, and include those devices shown throughout the Figures (or not shown but described), and reference numerals (or no reference numerals) associated with the devices are meant to be exemplary and not limiting. Further, the programs that run the smart devices or the external computer systems can be located at one or more smart devices or external computer systems, and work together or separately, and reference numerals (or no reference numerals) are exemplary and not limiting.)

But as the trend towards increased miniaturization of biotechnology and computers continues to expand, current limitations will in all likelihood diminish, so that in the future smart microbots and nanobots will possess the potential to modify their form and function as well, in a manner similar to those capabilities more akin to larger size smart medical devices.

But unlike in conventional cell biology where a given stem cell can differentiate into different cell types in an all or nothing manner, the differentiation of multi-functional smart medical devices can be defined in more subtle non-exclusive ways, by adding, subtracting, or modifying a specific individual function, while maintaining other pre-existing functions.

This imparts the ability of a smart medical device to continuously and dynamically alter its interactions with the host medical environment in an adaptive manner, based upon immediate and ever-changing biologic conditions. Conventional medical devices which are static and incapable of structural and/or functional change can now become dynamic facilitators of biologic interaction, for both diagnosis and therapeutic intervention.

A variety of ways can be employed to effect this change in biotechnology, which will be described in detail herein. These changes can occur through software and/or hardware modification and can be done at the level of the entire device, a portion of the device, or any of its embedded subcomponents. In addition, a unique and novel application can occur through biologic symbiosis, in which microbots and nanobots can combine with the host's cellular topology to create hybrid smart devices, in which combined molecules (i.e., nanobots and host cells) can interact to create new functional structures with unique functionality not available by either of the components acting alone.

### B. Changing Smart Device Functionality: Software

Smart device functionality is a combination of both its intrinsic hardware and software. Unlike hardware modification, which requires physical change in the form or structure of a given smart medical device, a software change may not change a given smart device's physical makeup.

Unlike most conventional medical devices, in one embodiment, in vivo smart medical devices possess the ability to perform such software changes without physical extraction from the host subject. In addition, in one embodiment, the software changes being implemented need not be solely under the direction of an authorized human third party but may instead be directed through intrinsic artificial intelligence (AI), which directs the software change based on real-time data collection and analysis. As a result, time sensitive dynamic changes which often occur in acute and/or critical medical conditions may be dynamically addressed in situ, without time sensitive delays.

Whereas conventional medical technology provides software updates on an ad hoc basis, often reacting to quality assurance issues, the present invention offers the opportunity of providing new and/or revised software in real-time and directly in response to real-time data analyses. In one embodiment, these in vivo smart medical device software changes are possible due to the incorporation of miniaturized microprocessors and bidirectional wireless communication capabilities directly into smart devices, along with the ability to directly communicate with an array of computers both within and outside of the host subject (see the incorporated patents/applications).

In one embodiment, as medical data intrinsic to the host subject is being continuously collected and analyzed by the program run on internal and/or external microprocessors, the data can be correlated with larger extrinsic medical databases by the program to enhance real-time diagnosis and treatment options. In one embodiment, the in vivo smart medical devices can also communicate with other smart devices which are located throughout the host subject, in both fixed and dynamically changing anatomic locations, to dynamically enhance disease detection and intervention.

In addition to software modifications related to quality assurance and quality control concerns, in one embodiment, new software may be deemed necessary in order to enhance, modify, or reduce a given smart medical device's functionality. Suppose, for example, real-time data analysis identifies the presence of a new emerging host pathology, which may not be optimally diagnosed and/or treated with existing in vivo smart device functionality. While the smart device components for such diagnosis and/or treatment may be available, the software required for new and/or enhanced functionality may be missing and/or deficient. In such an embodiment, the data becomes the driver for changing the functionality of a given in vivo smart device through new software downloads.

In one embodiment, the real-time data driving such a software change may come from a variety of sources, which include (but are not limited to) the following:
1. Internal smart device data analysis by the program (i.e., data derived by the smart device in question).
2. Analysis by the program by another smart device.
3. Analysis by an extrinsic computer program on the host network.
4. External input by an authorized third party (e.g., physician).

In the first three cases listed above, artificial intelligence (AI) (described below) provides the ability to collect and analyze dynamic real-time host data and determine the appropriate diagnostic and/or therapeutic interventions.

In order to illustrate how smart device software changes can be implemented in real-time to effect changes in smart device functionality, a few relevant examples are presented.

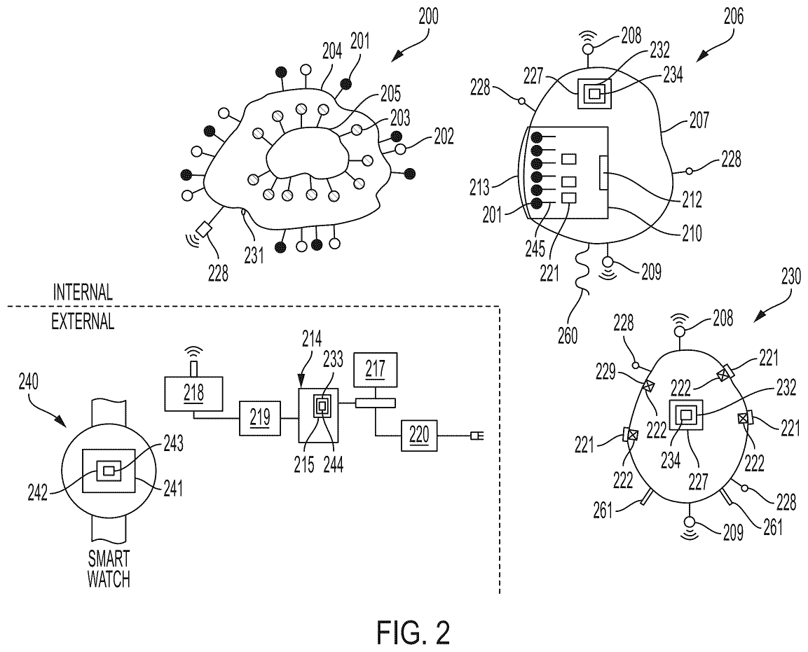

In one exemplary embodiment, take a smart nanobot (i.e., device 230, see FIG. 2) whose primary function is to acquire visual data of the local environment for the purpose of anatomic mapping and localization functions (as described in the incorporated patents/applications). Now suppose the host subject (not shown) experienced a sudden unexpected rise in body temperature which serves as a trigger for an acute infection or inflammatory response. There are a number of mechanisms in which the elevated host body temperature can be identified.

In one embodiment, an external smart device 240 (see FIG. 2), such as a smart watch 240, which has a microprocessor 241 with memory 242, can have its program 243 record the temperature reading from the host body, into memory 242 or data storage. The recorded smart watch data is in turn automatically transmitted by the program 243 to a host-specific medical database (i.e., an external database at a computer system—not shown) where if confirmed by the program by comparing additional readings, sends a message to an external centralized computer system 214 microprocessor 215 and program 244, the message which is recorded in database 233, for further steps.

Alternatively, in one embodiment, the elevated body temperature may be recorded internal to the host subject, by an in vivo medical device (e.g., smart catheter or circulating nanobot 230 with temperature sensors 221). In either case, the data measurements are transmitted by the program 243 to a centralized database (i.e., external) and continuously analyzed by the program 243, 234 to assess data progression.

In one embodiment, the ensuing actions by the smart device 230 are in part dictated by temperature trending analysis performed by the program 234. If the continuous data collection by the sensors 221, when analyzed by the program 234, validates the elevated body temperature and shows an incremental rise commensurate with an acute inflammatory response (by correlating with predefined temperature analyses), an intervention is triggered by the program 234 to determine both the underlying etiology and location of the offending agent.

In one embodiment, in order to accomplish this new data-driven directive, a number of smart device actions may take place by the program 234 in order to identify the location and etiology of the temperature increase. Some of these may require changes by the program 234 in smart device functionality. In one embodiment, a number of representative responses by the program 234 may include the following, which may be carried out by the program 234 (and/or the user):
1. Change in frequency and/or duration of temperature data collection.
2. Activation of embedded miniaturized technologies (e.g., temperature biosensors 221), which are currently turned off.
3. Deployment of new in vivo smart devices.
4. Reactivation of smart devices which are currently in a dormant state.
5. Activation of communication protocols by the program between in vivo smart devices (to coordinate device interaction).
6. Revision of existing device software protocols (to modify functionality).
7. Transmission of new software to introduce new device functionality.

In one embodiment, the net result is that temperature data may now be collected and/or communicated using the program 234, by a number of in vivo smart devices and/or components of the present invention. By the program 234 of the present invention modifying smart device and/or smart component software, the intrinsic functionality (and subsequent roles) of these smart devices 230 can be modified in accordance with host medical data change. In the event that the new and/or revised action being taken by the program 234 is only required temporarily, in one embodiment, a new transmission can be performed by the program 234 following successful completion to return the smart device 230 and/or component to its previous state. As a result, the software modifications are completely reversible, if needed.

One of the unique embodiments of the present invention is the ability to anatomically localize the source of pertinent data. This can be accomplished in a variety of ways including, in one embodiment, a triangulation of data by the program 234 (or program 244) from multiple in vivo smart devices (i.e., device 230) (which can be stationary and/or mobile), or real-time anatomic localization by the program 234, 244 of actively circulating smart nanobots. This unique ability of the program 234, 244 to anatomically localize data sources enhances the ability of the present invention to direct targeted smart device response for both diagnosis and treatment.

In one embodiment, the ability of the program 234, 244 of the present invention to perform continuous real-time data collection and analysis by individual and/or multiple smart devices working in coordination with one another provides for the creation by the program 234, 244 of trending analyses, which may be of particular importance in determining the importance of a given disease process as well as the urgency and specific type of treatment responses. Once again, in one embodiment, existing in vivo smart devices and/or components may be recruited by the program 234, 244 in both diagnosis and/or treatment through software transmissions which may modify smart device usage and functionality.

Another unique embodiment of the present invention is to modify a given smart device's functionality in accordance with anatomic location. Suppose, for example, real-time data and analysis by the program 234, 244 determines early-stage disease at a specific anatomic location (e.g., superior segment of the right lung lower lobe). The software transmission to the smart device (e.g., nanobot 230) may not only modify the functions of a given mobile smart device and/or components, but also dictate the specific anatomic location at which the action being taken by the program 234, 244 is performed. At the same time, the timing and frequency of the actions to be performed by the program 234, 244 may also be modified based on the software program directives.

Returning to the case study example where an elevation in the host subject body temperature was recorded in data storage and verified by program analysis (the program can be run internally at the nanobot 230, or externally at one or more computer systems 214, 240), in one embodiment, a number of smart device 230 actions may subsequently take place, for both diagnosis and treatment. From a diagnostic perspective, a number of clinical questions may arise, including but not limited to the following:
1. What is the underlying etiology of the temperature elevation?
2. Where is the anatomic location of origin?
3. What is the extent of inflammatory response (i.e., local, regional, or systemic)?
4. What type of host biologic response is taking place?
5. What potential role do existing in vivo smart medical devices play in diagnosis and/or treatment?
6. What type of smart device functionality is currently lacking, which could in theory facilitate improved diagnosis and/or treatment?

In one exemplary embodiment, suppose the existing in vivo smart devices include a tracheostomy tube, vascular catheter (i.e., catheter 300), and circulating nanobots (i.e., smart device 230). Embedded biosensors (i.e., sensors 221) within the walls of the circulating nanobots provide data such that the program (internal to the particular smart device, in this exemplary embodiment, nanobot 230) identifies the most likely source of fever is an acute inflammatory process within the superior segment of the lower lobe of the right lung as determined by anatomic mapping of the elevated temperature data recordings by the program 234.

One role of the existing smart nanobots 230 is to create a 4-dimensional anatomic visualization map through continuous signal transmission as the nanobots circulate throughout the host subject, as described in the incorporated patents/applications. But with the newly acquired data of an acute inflammatory response within the right lung, additional data is required to determine the specific etiologic agent. In one embodiment, one potential response aimed at gauging the host subject's biologic response would be to convert some of the circulating smart nanobots from anatomic localizers to white blood cell (WBC) trackers, since WBCs routinely migrate to the site of an acute infection, such as pneumonia.

In one embodiment, this functional differentiation of smart nanobots (e.g., nanobot 230) can be accomplished through the transmission of software, to update the program, which updated program (i.e., program 2340 can effectively modify the operation of the miniature video devices (i.e., at position 229) embedded within the nanobots' walls, so that they can now record and save in storage (e.g., memory 232), as well as analyze WBC movement within their proximity. In order to assist in this process, in one embodiment, the speed of travel and their range of movement of the nanobot 230 is restricted to the anatomic confines of the right lower lobe.

The net effect is that smart nanobots which had been originally tasked with rapid mobility and continuous travel with anatomic mapping of the entire host anatomy are now effectively converted into specialized smart nanobots whose program (i.e., program 234) records, tracks, and analyzes WBC movement within the narrowly restricted confines of the designated anatomic location of interest (i.e., superior segment of right lung lower lobe).

In one embodiment, as this new data is acquired by the program (e.g., program 234), a new and specialized right lower lobe WBC visualization map is created by the program, which quantifies the host subject's biologic response to infection, which in turn can be correlated by the program with body temperature. In one embodiment, the combined data of WBC quantification and localization, along with continuous temperature recordings provide new data-derived analyses of infection severity, anatomic location, temporal extension, and treatment response. These new analyses are the direct result of creating an entirely new class of smart nanobots, through software transmission alone.

In the exemplary embodiment, once the infection has been successfully diagnosed and treated by the clinician, the smart nanobots can now be reconverted by the program from WBC trackers back to generalized anatomic localizers through wireless software transmission, which returns the smart nanobots back to their original functional state.

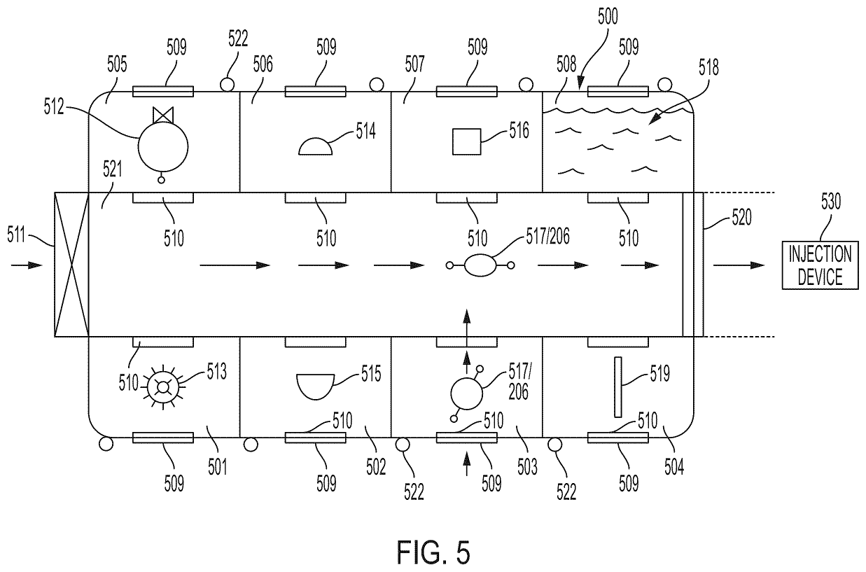

In another exemplary embodiment, suppose the smart tracheostomy tube contains a series of miniaturized drug storage devices (i.e., see FIG. 5, reservoirs or compartments 501-508) within its walls 523, along with an injection device 530 for local drug delivery. Once the right lower lung infection has been diagnosed (e.g., by culture and sensitivity of a collected sputum sample within the right lower lobe bronchus), the appropriate drugs of treatment can be determined by the clinician. In this particular case, one type of drug reservoir contained within the smart tracheostomy is a cephalosporin, which is determined by the clinician to be therapeutic in response to the offending bacterial agent.

In the incorporated patents/applications, the ability of smart medical devices to autonomously navigate and alter (i.e., compress, detach into components) their physical structure was described in detail. Given this ability, selected smart components embedded within the smart tracheostomy tube containing the cephalosporin drug reservoirs and injection devices can detach from the native tracheostomy tube upon program 244 (or internal program) and/or user instruction.

In one embodiment, in the event that the smart components are capable of autonomous navigation, they could theoretically navigate independently to the infection site. Alternatively, these individual smart components could coalesce to form a new smart device. A third possibility may include a smaller mobile smart device (e.g., smart microbot) serving as a substrate in which the smart components can embed and use as a transport vehicle to arrive at the anatomic location of interest.

In one embodiment, whether these smart components act independently or in conjunction with other smart components and/or devices, they functionally form a new class of smart components and/or smart devices, which can download corresponding software through wireless transmission. In one embodiment, the downloaded software can contain a variety of operational information which provides the smart components with the directives and artificial intelligence (AI) required to complete their mission. This represents another example of how smart in vivo devices and/or smart components can be effectively transformed into an entirely new class of smart devices from the parent smart device, through software program transmission alone.

In another exemplary embodiment, in the setting of acute life-threatening hemorrhage, where the host subject is physically removed from a medical care facility (e.g., penetrating trauma of soldier in the battlefield). In such a case, the acute traumatic event would be accompanied by sudden and precipitous hemodynamic instability (as determined by an increase in heart rate and/or drop in blood pressure, which can be recorded in an internal database by the program of the smart vascular catheter or by the program of an externally located smart watch).

For the circulating smart bots which were previously tasked with continuously mapping host anatomy, in this exemplary embodiment, the circulating smart bots could end up taking on a new role in response to the acute bleeding episode. Instead of generalized anatomic mapping, the smart bots could now be assigned by the program 244, 234 the task of identifying, localizing, and characterizing the specific bleeding source. In order to do this, in one embodiment, the smart bots will continue to circulate throughout the host vascular system and continuously send and transmit signals identifying their location over time, but with new software program updates, they will simultaneously collect and store real-time data related to localized change in vascular anatomy, pressure, and flow velocity for program analysis.

In essence, these smart bots have been transformed from a role of anatomic mapping to one of surveillance and detailed mapping of both vascular anatomy and pathology. In one embodiment, if the smart bots localize the anatomic location and severity of bleeding based upon their continuous data collection, another new software program upgrade may be transmitted from an external computer system 214 and received by the smart bots 230, further modifying smart bot functionality. In one embodiment, this new role becomes one of a smart bot aggregator, which includes the smart bots aligning with thousands (or even millions) of other smart bot aggregators at the specific bleeding site. In doing so, these aggregates of smart bots now form a vaso-occlusive macrobot compound which serves to therapeutically obstruct the damaged blood vessel at its bleeding site and reducing active bleeding.

In one embodiment, another new role assigned to the smart bot may be that of a smart bot facilitator. Rather than simply becoming a part of an aggregated macrobot, the software program update it receives may provide it with artificial intelligence (AI) required to facilitate the thousands of aggregating smart bots, in essence providing input to these bots as to where to position themselves within the aggregated mass being created.

In one embodiment, the transformation to this new role was solely accomplished through real-time software program upgrades with continuous feedback being provided to the program for analysis by continuous real-time data being collected by smart bots, in addition to other in vivo smart devices.

The examples provided herein illustrate how software program modifications alone can inherently change smart device functionality. But the transformations which take place need not be permanent and can be readily altered in accordance with the constantly changing host requirements and health status, which is continuously being analyzed through real-time data collection and analysis by the program.

In one embodiment, the smart bots can continue in their new functional state, revert back to their previous state, or take on an entirely new role as needed. This highlights a unique feature of the present invention which ironically is not available in pluripotent stem cells. While stem cells have the ability to differentiate into a variety of new cell types, the differentiation is unidirectional. Once a stem cell is transformed into a platelet, it cannot revert back to its original state as a stem call.

Smart devices, on the other hand possess the ability to continuously undergo modification in form and/or function (which in this case was through software program change alone), in an entirely reversible process. Since the smart devices and/or smart components are in vivo and responding in real-time, the functional changes can take place in a matter of minutes (or even seconds), rapidly changing the time course of medical diagnosis and/or treatment.

### C. Changing Smart Device Functionality: Hardware

While the previous exemplary embodiments illustrated how various types of smart devices can be functionally differentiated into new and different types of smart devices through software modification, similar smart devices transformation can also be accomplished through changes in smart device hardware.

In the present invention, one important concept for smart device differentiation is the ability to modify function in accordance with an immediate need in real-time. Rather than having to wait an inordinate amount of time for external data collection, analysis, decision making, and intervention (which is largely done ex vivo), the present invention provides a mechanism for immediate in vivo intervention based upon anatomy, pathology, criticality, and available resources.

Unlike software only smart device differentiation, which involves no true structural change to the original smart device, hardware driven device differentiation of the present invention is accompanied by physical change in smart device structure, form, and/or content. Another embodiment of the present invention includes combined software and hardware differentiation, in which change in device functionality and performance is driven by both software and hardware modifications.

In one embodiment, the simplest form of smart device hardware modification can occur at the level of the miniaturized subcomponents contained with a given in vivo smart device. These subcomponents can be added, removed, or modified to effect functional change in the activity and/or performance of the smart device.

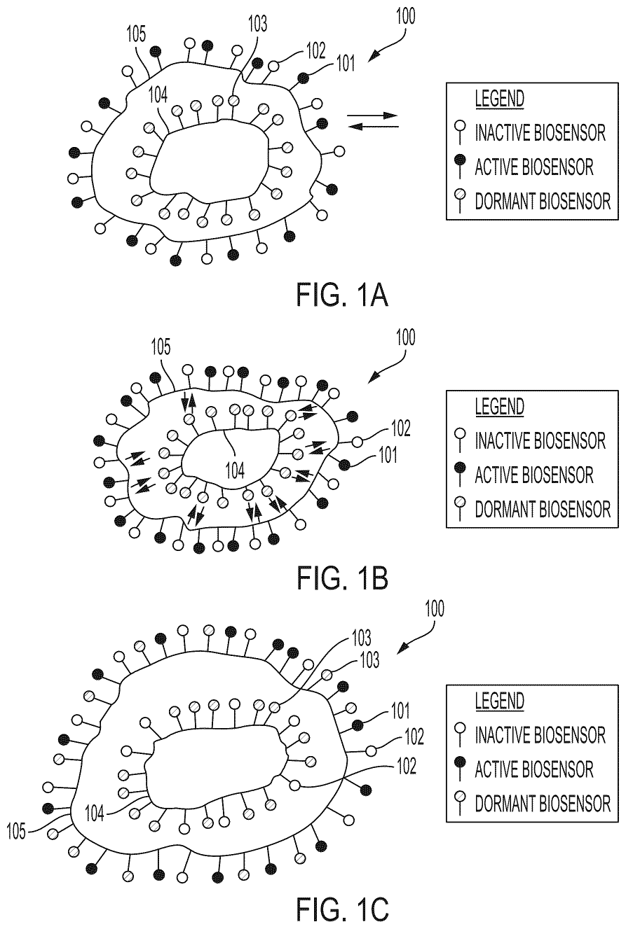

In one exemplary embodiment, a smart device (e.g., diagnostic biosphere 100 (see FIG. 1A), which is a form of microbot), containing a number of miniaturized biosensors performing different types of bioassays, is tasked with travelling throughout the host anatomy and continuously performing bioassays for disease surveillance.

Since the smart device of the present invention is tasked with disease surveillance, the biomarkers (and their corresponding bioassays) which are routinely performed, are in large part generalized and relatively nonspecific in nature. One example is that of C reactive protein (CRP), which is a highly sensitive, yet nonspecific biomarker for inflammation. The advantage is that it will be highly accurate in identifying early signs of inflammation, but poor in specifying the underlying etiology. As a result, it serves as an excellent early warning sign, which is ideal for disease surveillance. The downside is its lack of specificity, requiring more in-depth analysis, which would be the province of other more specific biomarkers.

In the previous exemplary embodiment, of pneumonia within the lower lobe of the right lung, as the smart device 100 circulates through the host vascular system, it begins to record progressively increased levels of elevated of CRP (which would be recorded at its highest levels in the blood vessels supplying the involved right lower lobe) in the external database 233.

As additional data is recorded in the data storage 233 by the program 244, the increased levels may exceed a preprogrammed level, which raise the concern for infection, such that additional bioassays more specific to infection are required by the program 244 and/or user, such as procalcitonin (PCT). But the corresponding PCT biosensors 101 are not readily available in the existing smart device architecture, whose primary role is designed for surveillance, as opposed to diagnosis.

In this particular exemplary embodiment of the smart biosphere or biosensor 100 of the present invention, the smart biosphere 100 includes an inner layer 104 and outer layer 105 of biosensors, with a large number of biosensors 101-103 contained within different locations/layers of the biosphere 100 (see FIGS. 1A-1C). Some of these biosensors 101 are active, others 102 inactive, and others 103 may be readily available but in a dormant state, which means they can become activated through a single software program command (FIG. 1A). By doing so, the functionality of the smart device 100 changes in direct response to clinical need (i.e., software-mediated device differentiation).

However, in a different exemplary embodiment of the present invention, the biosensors 103 used for assaying PCT are physically located in a separate and inaccessible location (i.e., inner layer 104) within the biosphere 101 architecture. In order to activate PCT bioassays 103, the corresponding biosensors 103 must be physically repositioned from the inner depths of the smart device 100 to the surface 105, where they can now become activated and perform bioassays.

In one embodiment, in order to do so, a rotational mechanism within the smart device 100 architecture may be activated by the program 244, which in effect will shift the position of the PCT biosensors 103 from an inner (and inactive) compartment or layer 104 to the smart device 100 surface 105, where they can become active (see FIG. 1B). Thus, the outer inactive biosensors 102 change position with the inner (dormant) biosensors 103. Once this biosensor 100 rotation has been successfully completed (FIG. 1C) as determined by the program 244, the biosensor 100 can undergo a series of quality control (QC) steps by the program 244 to verify proper performance, and then activated by the program 244 to begin performing PCT assays.

So, in this exemplary embodiment, the hardware of interest was physically contained within the smart device 100 and simply required positional change and activation by the program 244 when the predetermined requirements are met, prior to becoming functional. Thus, a new and functionally different smart biosphere 100 is created. This process is reversible and subject to continuous change based on changing biodata and the need of the user.

In one embodiment, where the subcomponent(s) (i.e., biosensors 101) of interest are not physically located in the host smart device 100 and instead required introduction from an external source, such as another in vivo smart device 100 or device depot (which will be described below), then an artificial intelligence (AI) or human-directed intelligence may identify an entirely different and more specialized bioassay which would enhance diagnostic capabilities, in the form of interleukin 6 (IL-6). But since in this exemplary embodiment this particular biosensor 101 is not currently available within the smart biospheres 100, it must therefore be sourced from an external location.

In this embodiment, the smart device component (i.e., biosensor 201) (see FIG. 2) is introduced from an external source (i.e., smart device 206) to biosphere 200, because biosphere 200 lacks the number or kind of biosensor desired (i.e., biosensor 201) among the inactive biosensors 202. In this scenario, the desired biosensor is not one that can be activated by switching dormant biosensors 203 on inner layer 205 to outer surface 204 and activating them. Instead, they are introduced into biosphere 200 from an external device.

In one embodiment, the smart device 200 in which the new subcomponent (i.e., biosensor 201) is being introduced (e.g., smart biosphere 200), may be referred to as the “receiving device” 200 and the source of the subcomponent (i.e., biosensor 201) being introduced may be referred to as the “donor device” 206. In one embodiment, the donor device may take the form of another smart device 230 such as a nanobot or smart medical device (see the incorporated patents/applications) or that of a less developed “device depot” 206, which in effect, is mainly a storage device 210 in which various smart device subcomponents (i.e., biosensors 201, sensors 221) are housed within the host body 207.

In one embodiment, the smart device/device depot 206 includes a signal emitter 208 and signal receiver 209, which navigates the patient body based on, for example, continuous feedback of transmitted signals from other medical devices (i.e., smart device 230), an external transmitter/receiver 218 connected to a computer system 214, or from transmitted signals from within a target location (not shown). The smart device/device depot 206 may include, at least, one or more of a camera (not shown-see the incorporated patents/applications), propulsion and steering mechanisms (not shown-see the incorporated patents/applications), sensors 221, energy storage devices (not shown-see the incorporated patents/applications), anchoring devices (not shown-see the incorporated patents/applications), and computer electronics systems 227, including memory 232 and program 234 (microprocessors, microcontrollers) (see the incorporated patents/applications).

As shown in FIG. 2, in one embodiment, the smart device/device depot 230/206 can be controlled from an external computer system 214 having a microprocessor 215 having an internal memory 233 and program 244, a controller 219 (internal or external), connected to a display 217 for a user. In one embodiment, the computer system 214 is connected to and controls an external charging source 220 for the smart device/device depot 230/206. In one embodiment, the computer program 244 (run by microprocessor 215 and/or by internal smart device/device depot 230/206 microprocessor systems 227) monitors all aspects of the smart device/device depot 230/206 operation, including the transmission/receipt of signals, energy usage, propulsion, steering, sensor operation, etc. (see the incorporated patents/applications).

In one embodiment, the transportation of the subcomponent (i.e., biosensor 201) of interest may take place in a variety of ways. If the donor device 206 is mobile, it may travel to the location of the receiver device 200, in order to facilitate subcomponent transfer. Alternatively, if the receiving device 200 is mobile, it could travel to the location of the donor device 206 (if, for example, the donor device 206 is not mobile).

Another option for subcomponent transfer may take the form of a delivery smart device (analogous to a tow truck), which serves as a third-party delivery device for transporting the subcomponent of record from its donor smart device 206 to receiving smart device (i.e., biosphere 200) (for example, an anchoring device deployed from one smart device can attach to another donor smart device and be towed to the receiving device) (see the incorporated patents/applications).

In one embodiment, once the subcomponent transport has been successfully completed, a variety of methods may be used to add/install the miniaturized subcomponent (i.e., biosensor 201) into the receiver in vivo smart device (i.e., biosphere 200), such as an installation device 212 (i.e., a hydraulic pump, air gun, or mechanical ejection arm or screw), via opening 213. Thus, a variety of methods may be used suitable to the smart device/device depot 206 (as donor device), and the smart device/biosphere 200 (as receiving device), so that miniaturized subcomponents 201 can be transferred to a receiving in vivo smart device 200 from a donor device 206. Smart device design and construction can be deliberately planned to accommodate such hardware modifications.

In the present exemplary embodiment of the smart biosphere 200 containing a variety of active biosensors 201, smart device donor 206 design can incorporate empty compartments 210 to accommodate physical introduction of new biosensors (not necessarily the same as biosensors 201 in biosphere 200) into the outer wall 204 of the biosphere 200, where the biosensors 201 can become activated by the program 234/244, once they are introduced and tested for quality control by the program 234/244. In doing so, the smart device 200 changes its inherent functionality through the physical addition of new device subcomponents.

In one embodiment, in order to facilitate the introduction and correct orientation of a new subcomponent within the receiving smart device, a variety of strategies may be employed to facilitate the new subcomponent placement. One exemplary embodiment would include smart device 230/206 having an electronic beacon 228 or other type of navigational aid 228 to help the subcomponent (i.e., biosensor 201, sensor 221) identify and navigate to the specific position within the receiving smart device (i.e., smart device 206/230 or biosphere 200) in which it will be incorporated. By possessing the ability to transmit and receive signals between donor/receiving devices and their respective smart subcomponents, continuous communication can take place between the receiving smart device, the donor smart device, and/or the specific smart device subcomponent/s (i.e., biosensors 201, sensors 221) being integrated into the receiving smart device.

In one embodiment, an additional feature which can be incorporated into the smart device integration process is that of a locking mechanism 222 (see smart device 230, for example) which provides the means with which a newly incorporated subcomponent (i.e., sensor 221) can be properly secured into the receiving smart device architecture (for example, at sensor position 229 in another smart device) once successful navigation of the smart subcomponent (i.e., sensor 221) has been completed. In one embodiment, once the navigation has been successfully completed as determined by the program 234/244, the locking mechanism 222 can be activated by the program 234/244 (after the appropriate security features have been verified by the program 234), and the subcomponent (i.e., sensor 221) can be physically secured in its new location by the smart device. At the same time, if a given smart component (i.e., sensor 221) is to be removed, the same locking mechanism 222 can be released, thereby allowing a given smart component (i.e., sensor 221) to be removed or repositioned from its original location.

In one embodiment, the present invention includes important security features run by the program to ensure that all modifications in smart device and/or smart component structure, form, and functionality are appropriately vetted and commensurate with the host subject ever-changing medical status. In the event that the security protocol was not properly completed or deemed to be violated by predetermined program rules, an emergent shutdown feature could be activated by the program (i.e., program 244).

In addition to transport of the desired subcomponents via navigation of a donor or transfer smart device, in one embodiment of the present invention, the smart device subcomponent (e.g., biosensor 201) can also independently navigate itself to the location of the receiver smart device, if the device subcomponent was to possess its own internal autonomous navigation system (e.g., tail 245, etc.) as described in the incorporated patents/applications and priority documents). In this embodiment, the subcomponent could self-navigate to the location of the receiving device, go through the verification process and security protocols per program 244, and physically insert itself at the designated location within the receiving device architecture (i.e., biosensor 201 propulsion/steering mechanism, tail 245, would be able to reach beacon 228 and insert itself at position 231 of biosphere 200).

Once the appropriate quality control (QC) protocols and testing by the program 244 of the computer system 214 has verified proper functioning of the subcomponent (i.e., sensor 221 inserted at position 229) and its communication with the receiving smart device's internal computer system 227, the subcomponent (i.e., sensor 221 in this example) can be activated by the program 234/244 and begin routine function. Upon completion, the receiving smart device (i.e., device 230) possesses new and/or modified functionality, effectively differentiating itself into a functionally different type of smart device.

In one embodiment, in the same manner that subcomponents can be added to a given medical device to enhance and/or modify its inherent functionality, device subcomponents can also be removed, which also serves as a method by which device functionality can be modified.

In one embodiment, in the previously cited example of the smart biosphere 100 which had IL-6 biosensors added to its architecture, suppose a different class of biosensors (e.g., cytokine bioassays) was removed in the event that they no longer were deemed necessary. This would change the inherent functionality of the smart biosphere 100 and redefine its diagnostic capabilities. With the newly created capacity caused by the removal of these cytokine biosensors, in one embodiment, an entirely new type of subcomponents could be introduced (e.g., miniaturized lasers, cutting device, tool—not shown, but see incorporated patents/applications), which completely transform the functionality of the smart biosphere from that of purely diagnostic to that of combined diagnostic and therapeutic. The bottom line is that a given smart device's internal architecture and components are designed to be flexible and dynamic, thereby allowing the smart device functionality to be reversibly modified in accordance with need.

In this exemplary embodiment, the various hardware modifications described can be completely reversed by the program 244 once the acute medical emergency has been successfully diagnosed and treated, effectively returning the smart biosphere 100 to its original surveillance state. This reversibility in smart device form and function is an important feature of the present invention since it provides a mechanism for smart device repurposing, without having to consistently introduce new smart devices and/or components.

In one embodiment, another advantage of the ability of the present invention to add and subtract smart device components is that it may both increase the practical utility, and prolong the effective lifetime, of a given smart device. In one embodiment, if a given in vivo smart device maintains its structural and functional integrity over an extended period of time, its utility is enhanced and the requirement for smart device turnover is reduced. Miniaturized subcomponents which have become obsolete, broken, deficient in function, or are simply no longer needed can be removed by the program 244 and replaced by new and/or different components deemed more necessary.

One aim of the present invention is to minimize technology obsolescence of in vivo smart devices, particularly those which are introduced to be permanent or semi-permanent in duration (e.g., smart cardiac pacemaker, prosthesis, or implantable pump). By designing the smart devices to have replaceable parts or subcomponents, the effective lifetime of the core smart device may be prolonged. At the same time, innovation in subcomponent technology, allows these smart device subcomponents to be replaced when deemed to be deficient in form or function to its new and improved counterparts.

The present invention also creates methods by which larger device components (containing numerous individual subcomponents) can be added, subtracted, and/or reconfigured. In this embodiment, individual segments of a smart device can be added, subtracted, and/or modified to alter device functionality, as well as create new and separate smart medical devices.

Segmental architecture of how smart devices can be altered is described in the incorporated patents/applications. In one embodiment, smart devices may be constructed and connected to one another using an articulated design, thereby allowing for individual segments of a given smart device to be easily removed or added from the native smart device.

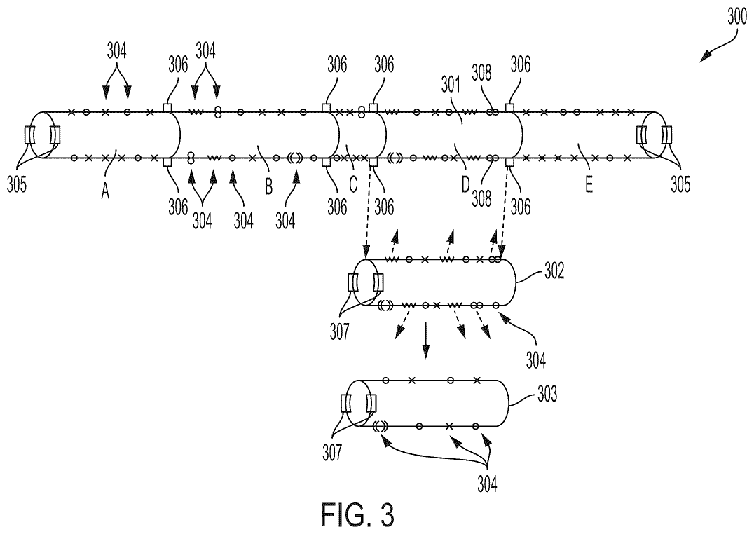

In an exemplary embodiment, a smart vascular catheter 300 may be constructed in a linear fashion, using five articulated segments (segments A-E, each of which contains a number of individual miniaturized subcomponents 304 marked with various symbols) (see FIG. 3). The construction of the smart catheter 300 is done in a way that provides different levels of functionality within each individual segment A-E, so that the total utility and functionality of the smart device 300 is a sum total of each individual segment A-E. In the event that one segment (e.g., segment D) of the smart device 300 was to be removed or replaced, the functionality of the newly modified smart device 300 would be defined by the remaining segments (e.g., segments A-C and E and corresponding subcomponents 304) and/or the newly modified segment(s) (i.e., segment 302 or subsequently segment 303).

In an exemplary embodiment, segment D of the smart vascular catheter 300 is removed by a locking mechanism 306 (as described above) being unlocked (e.g., unlatched, released by an attachment/release means), and the segment 302 transported by its own propulsion and/or navigation systems 307, or towed by another smart device (described above—not shown) to a different anatomic location in the left lobe of the liver, where it is used for monitoring of cellular activity and focal drug delivery in the treatment of liver cancer (i.e., hepatocellular carcinoma). Further, as shown in FIG. 3, this segment 302 can be modified by program 244 instructions to achieve a new functional unit 303 when individual components 304 are added or subtracted. Of course, the smart vascular catheter 300 can be reconstituted and secured by the locking mechanism 306 without segment D, when moved together under its own propulsion and navigation systems 305 (e.g., chemical, mechanical tails/flaps 260, 261, etc.).

In this exemplary embodiment, the drug delivery subcomponents 308 within the original smart vascular catheter 300 were the only therapeutic subcomponents 304 in its original architecture. As a result, the removal of segment D now transforms the smart vascular catheter 300 from that of a combined diagnostic and therapeutic device to a solely diagnostic device.

In another embodiment of the present invention, the same smart vascular catheter 300 is deconstructed itself into three separate and distinct smart devices, with the first smart device containing segments A and C, the second smart device containing segments B and D, and the third smart device containing segment E, along with an additional subcomponents (not shown) obtained from a smart drug infusion port which was located in the left portal vein.

The net result in this embodiment is that one original in vivo smart device has been transformed into three separate smart devices, each with its own unique structure, content, and functionality. But the deconstruction or disassembly of smart medical devices 300 need not be restricted to those having an articulated architecture. Any and all portions of a given smart device 300 has the potential to detach a segment 302 of itself from the native smart device 300 and be used independently or in combination with other smart devices 300 and/or their segments 302/303.

In another embodiment of the present invention, the ability to construct in vivo smart medical devices from a number of disparate components/segments, is analogous to Lego® building blocks. This embodiment may be in response to an emergent and highly critical situation, where time is of the essence, thereby limiting the practicality of introducing new smart medical devices to the host subject. In such a scenario, a life-threatening condition would be identified by the user and confirmed by the user and/or program 244 through real-time data collection and analysis, prompting immediate response by the user and/or program 244.

In one embodiment, in the event that existing in vivo smart medical devices are not readily available to address the clinical emergency and introduction of new intact smart medical devices is not a practical option, then an alternative solution may be available in the form of newly constructed in vivo smart devices. In one embodiment, these may be created through incorporation of individual smart device components/segments into a newly created smart device in response to the emergent and acute clinical scenario. These will be denoted as “on the fly” or “impromptu” smart devices, in keeping with the time urgency and disparate manner in which they can be created.

In one embodiment, the subcomponents for these impromptu or extempore smart devices may come from a variety of sources including (but not limited to) implanted smart devices, mobile smart devices, in vivo device depots, externally located smart devices, or newly administered smart devices. In one embodiment, various components and/or segments of these smart devices may be aggregated and assembled in vivo to form the newly created impromptu smart device (see the incorporated patents/applications).

In one exemplary embodiment, to illustrate how these impromptu smart devices may be created, an acute life-threatening pulmonary embolus is located in the right main pulmonary artery of a patient. The occlusion of the right main pulmonary artery can be a cause of hemodynamic instability, which can lead to a number of abnormal biodata indicators including (but not limited to) increase in heart rate (i.e., tachycardia), increase in respiratory rate (i.e., tachypnea), and drop in blood pressure (i.e., hypotension); all of which can be readily identified by biosensors embedded within external (e.g., smart watch 240) or internal (e.g., smart vascular catheter 300) smart devices (i.e., biosensors 304 in FIG. 3).

In one embodiment, once this abnormal biodata is identified and verified (through continuous real-time data collection by the program 244), additional higher-level data is recorded by the program 244 in the data storage (i.e., memory 233) and analyzed by the program 244, with the goal of determining the underlying etiology. This can be accomplished by utilizing additional smart medical devices, which in this example would include both a smart implanted cardiac pacemaker and coronary artery stent (not shown).

In one embodiment, biosensors contained within these smart devices provide a number of additional biodata, including (but not limited to) the following:
1. Arterial blood gas measures (e.g., oxygen).
2. Cardiac chamber pressure and volumetric measurements.
3. Cardiac rate and rhythm.
4. Coronary artery perfusion.
5. Volume and pressure measurements of the major vessels outside of the heart (e.g., pulmonary arteries, thoracic aorta).
6. Bioassays (e.g., troponin, D-diner, thromboxane A2).

In one embodiment, with the additional biodata collected and analyzed by the program 244, the program 244 determines that the host subject is experiencing right ventricular failure (as demonstrated by increased ventricular size and diminished contractility), accompanied by progressively decreased blood pressure measurements, which would be indicative of impending shock, constituting a true medical emergency.

In one embodiment, at the time this data is collected and analyzed by the program 244, the host subject is in a State Park and unable to avail outside assistance and/or intervention. Although the biodata has been automatically transmitted by the program 244 to his/her designated healthcare personnel and emergency contacts via electronic means (i.e., text, fax, email, etc.), the estimated response time for emergency medical personnel is 35 minutes, which may be too late, given the criticality of the situation.

As a result of the time constraints, both diagnostic and therapeutic responses must rely on readily available resources, which in this case includes existing in vivo smart medical devices, which include the following:
1. Smart cardiac pacemaker.
2. Smart coronary artery stents.
3. Smart inferior vena cava (IVC) filter.
4. Smart venous catheter.
5. Smart circulating microbots and nanobots.
6. Smart device depot.

In one embodiment, in addition to stationary miniaturized devices and biosensors contained within the embedded smart cardiac pacemaker and coronary artery stents, mobile smart devices can also serve to collect real-time data throughout host anatomy. These migratory smart devices can take a number of forms and arise from various sources including (but not limited to) detached miniaturized components from embedded smart devices (e.g., smart pacemaker), smart components within mobile smart devices (e.g., autonomous smart venous catheter), or circulating microbots and nanobots. See the incorporated patents/applications for further explication.

In one embodiment, as discussed above, the function of these smart devices can be transformed and/or modified in conjunction with need, through changes in software and/or hardware. In one embodiment, as additional real-time data is collected and analyzed by the program 244, it is determined by program analysis that the cause of right ventricular failure and hemodynamic instability is a large occlusive embolus in the right main pulmonary artery measuring 5×3 cm. At the same time, the original source of the pulmonary embolus is deep venous thrombosis in the left subclavian vein (which happened to be the location of a prior vascular catheter, which had been removed three (3) weeks earlier). It was a segment of this deep venous thrombosis which detached and embolized to its current location within the right main pulmonary artery.

Given the acute nature and severity of the pathology, the program 244 and/or user determines that an emergent intervention is required if the host subject is to survive. In one embodiment, the primary intervention options presented by the user and/or program 244 include (but are not limited to) the following:
1. Pharmacologic vasopressors (e.g., dopamine).
2. Increased oxygen carrying capacity (e.g., infusion of artificial oxygen carriers).
3. Thrombolytic agents to dissolve clots (e.g., tissue plasminogen activator (tPA)).
4. Physical breakage of the embolus (e.g., rotational embolectomy).
5. Aspiration of the embolus (e.g., suction embolectomy).

Secondarily, it is important to address the primary source of the embolus (i.e., deep venous thrombosis), to prevent recurrent pulmonary emboli. In one embodiment, in addition to the intervention options listed above for the pulmonary embolus, another option would be placement of a mechanical filter (not shown) in the subclavian vein, similar to the smart filter located in the IVC of the host subject (which was placed to collect detached lower extremity venous thrombi).

In one embodiment, pharmacologic infusion can be readily performed by smart devices containing drug reservoirs (i.e., similar to compartment 210

in smart device 206 in FIG. 2) and infusion ports or needles (see the incorporated patents/applications). Physical dissolution of the embolus can be performed by the smart devices in a variety of ways, each of which requires its own unique smart device components, including (but not limited to) ultrasound-assisted thrombolysis (using high frequency ultrasound to penetrate the embolus and allow pharmacologic agents direct inner access), rheolytic embolectomy (using injection of pressurized fluid to macerate the embolus and then aspirate the broken fragments into a storage device (i.e., compartment 210)), and rotational embolectomy (using a rotating device to fragment the embolus and collect the fragments within a storage device (i.e., compartment 210)).

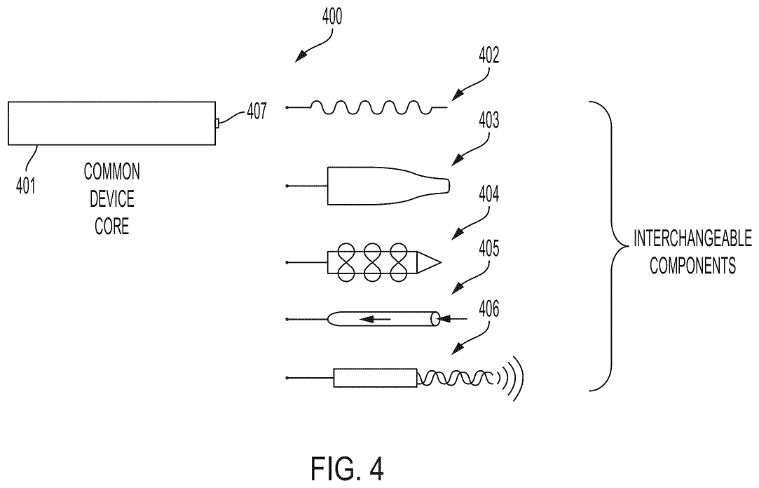

In this exemplary embodiment, the existing smart venous catheter 300 can serve as the primary receiving vehicle, by navigating itself under its own propulsion and navigation systems 305 to the right main pulmonary artery at the location of the occlusive embolus. Once there, miniaturized components 304 contained within the catheter 300 can perform the requisite actions. If the required components 304 are not originally contained within the catheter 300, they can be incorporated from other sources (e.g., a device depot 210). In order to prevent broken embolus fragments from travelling downstream and lodging within distal pulmonary artery branches, a variety of smart devices may be deployed (e.g., using umbrella or suction devices (see FIG. 4, described further below)) to collect and trap macerated embolus fragments (in a compartment 210) (see the incorporated patents/applications, where various grabbing mechanisms are used to trap and place items within a storage compartment of a smart device).

While existing in vivo smart devices can serve as donor sources for required smart device components, they can also serve as foundational sources for larger smart device construction. In the cited exemplary embodiment, the host subject has an existing smart IVC filter and is now in need of a subclavian vein filter to prevent formation of additional pulmonary emboli. The smart IVC filter can be deconstructed into individual subcomponents or segments which can now travel under their own propulsion/navigation systems, or be towed, and reaggregate in the subclavian vein (also, see incorporated patents/applications for examples). This deconstruction process can be done in toto or in part.

In an exemplary embodiment, suppose a temporary subclavian filter can be created using a fraction (e.g., 40%) of the existing IVC filter struts. This would allow the existing IVC filter to fractionally remain while simultaneously giving rise to the new subclavian vein filter being constructed. The present invention, thus, temporarily removes components of the IVC filter for the purpose of using these components to construct a new subclavian vein filter. Once the immediate crisis has been averted, additional components can be added from an ancillary source by the program 244 and/or user, to return both filters to their full size and complete state.

In one embodiment, while relatively minor and/or commonplace changes in health status can be accommodated by transforming or repurposing existing smart devices and subcomponents, major and/or unexpected changes in health status may require more specialized smart devices and/or components not readily available. Thus, in an emergent time-sensitive situation, alternative smart device and/or component sources may be required.

In one embodiment, one such source for additional smart devices and/or components would be an in vivo “smart device depot” (similar to depot 210), which is designed as a storage facility for specialized and/or redundant smart devices and/or components. These smart device depots can exist in in vivo or ex vivo forms. The locations of these in vivo device storage depots may be influenced by the physical size of the depot, host subject size, host subject anatomy, and smart device contents. Examples of possible device in vivo depot locations include (but are not limited to) subcutaneous soft tissue, intramuscular, intraperitoneal cavity, or intravascular.

In the exemplary embodiment, the host subject has a pre-existing history of lower extremity deep venous thrombosis (DVT), as evidenced by the indwelling smart IVC filter. As a result, the patient is at high risk for DVT and pulmonary embolus (PE), which in turn results in a number of in vivo smart medical devices and components specific to diagnosis and treatment of thrombo-occlusive disease. This would account for the presence of a number of related smart devices and components contained within the in vivo device depot. This embodiment illustrates how a host subject's medical history and/or disease predilection can be used by the program 244 to create in vivo device depots which are specific to each individual host subject. In some respects, they can be thought of as storage units, which contain a variety of smart devices and/or components pertinent to each individual host's medical profile.

In one embodiment, the present invention provides for the diversity of smart devices and/or components required for diagnosis and/or treatment for the bevy of potential pathologies and creates smart medical devices with interchangeable parts. By doing so, one could, in effect, create a series of smart device templates which can be functionally changed through the incorporation of different smart components, each of which can be selectively designed for a specific function, which can be context and user-specific. The multiple factors affecting a given host subject's medical history and predilection for disease (as listed below) can be used in creating a dynamic inventory for in vivo and ex vivo smart devices, specific to the individual host subject.

### D. Factors Affecting Smart Medical Device Design, Creation, and Inventory (Host-Specific Medical Profile)

1. Medical History.
2. Surgical/Procedural History.
3. Biotechnology.
4. Pharmacology.
5. Occupational History.
6. Social History.
7. Family History.
8. Environmental Exposures.
9. Genetics.
10. Medical Literacy.

In one embodiment, as new and continuous real-time biodata is collected and analyzed by the program 244, this process of smart device design, creation, implementation, and storage is continually updated by the program 244 and provided to the user, to reflect the changing healthcare dynamics within each individual host.

One exemplary embodiment which illustrates how smart devices with interchangeable components can be created and used, is the occlusive pulmonary embolus causing right heart failure and hemodynamic instability. Once the diagnosis, physical characteristics, and anatomic location of the pathology has been established through real-time biodata collection and analysis by the program 244, the next step is for the program 244 and/or user to create an effective intervention strategy, taking into account the diagnosis, host subject anatomy and medical status, time urgency, and available resources.

In this particular exemplary embodiment, the host subject is physically and temporally removed from external assistance, and given the life-threatening nature of the disease, must have intervention which is immediate and readily available. As such, the intervention strategy must be designed to take advantage of all readily available resources, in the form of accessible smart medical devices and/or components.

The present invention includes in vivo smart medical devices whose program(s) 234/244 are instrumental in establishing and defining the diagnosis. However, the present invention offers more than just intervention (i.e., therapeutic) options directed to local infusion of pharmacologic agents and relocation/creation of an intravascular filter to the origin of the embolus (i.e., subclavian vein). In fact, the sheer size, magnitude, and criticality of the pulmonary embolus and its clinical impact necessitates an intervention strategy focused on physical breakdown of the embolus, in order to reduce the mechanical burden on the failed right ventricle and re-establish pulmonary arterial blood flow.

Since mechanical breakdown of embolus requires highly specialized smart medical devices and components, and these would be unlikely to be readily available in the existing in vivo smart devices, the present invention makes these devices available in the case where a smart device storage depot (similar to depot 210) is present.

Given the past history of DVT, a smart device depot (e.g., depot 210) could contain smart devices and components dedicated to the treatment of thrombus and/or embolic disease. Obviously, the size and design of these smart devices and/or components would be specific to the anatomic region of interest, size, and composition of the thrombo-embolic disease process. As an example, occlusive disease in the brain would entail treatment of disease within a small caliber cerebral artery or vein, which are routinely millimeters (mm) in size. Alternatively, occlusive disease within the pulmonary artery involves a vessel measured in centimeters (cm). As a result, a ten-fold difference in anatomy size exists, requiring comparably sized smart devices and/or components.

In one embodiment, FIG. 4 presents a number of examples of smart devices and components specific to the treatment of thrombo-embolic disease. While having individual smart devices would certainly be an option, there are physical space requirements to store a large number of these smart devices.

In one embodiment of the present invention, the smart device 400 includes a central core 401 which would be relatively fixed (with a plurality of different size and structural options available to accommodate different anatomic regions). In one embodiment, the individual smart components—i.e., a drill component 402, an umbrella component 403, a rotating emblectomy device 404, a suction vacuum 405, and ultrasound transducer 406, for example—are responsible for intervention, and would be designed to be interchangeable, so that one smart device hub 401 could potentially give rise to tens (or even hundreds) of different smart interventional devices 400. In one embodiment, a smart device 400 option may provide ultrasound-guided thrombolysis (e.g., component 406), while others could provide rheolytic embolectomy (e.g., component 403), rotational embolectomy (e.g., component 404), aspiration thrombectomy (e.g., component 405), and thrombus fragmentation (e.g., component 402).

In one embodiment, at the same time, synergistic smart devices which are tasked with collecting thrombotic/embolic debris such as umbrellas (e.g., component 403), filters, and vacuums (e.g., component 405), could also be created using this interchangeable component model. As new interventional strategies are developed, corresponding smart components can be created, thereby expanding the number and diversity of smart device interventional (and diagnostic) options. In one embodiment, once a diagnosis is established and the various treatment options are analyzed (using an AI program 244, for example), the optimal smart devices are created by the program 244 incorporating the selected smart components (e.g., components 402-406) into the core smart device (e.g., component 401) at connection point 407. In one embodiment, a variety of standard systems could connect the components 402-406 to the core device 401, which include insertion and locking mechanisms 407 (i.e., latches, locks, adhesives, etc.).

Thus, the present invention provides a mechanism for creating multi-functional tools using a single core design. So, in summary, several different smart device hubs (i.e., central core 401), can accommodate a variety of interchangeable smart components (i.e., components 402-406), which can vary in size, function, and structure. In one embodiment, these interchangeable components can be added, subtracted, and/or replaced on an ad hoc basis, in accordance with the given medical circumstance. Once that specific smart device's mission has been completed, it can be returned upon command by the program 244, to its original location, which in this case may be an in vivo storage depot (e.g., depot 210).

The pluripotential smart device options of the present invention become almost endless and this provides a practical mechanism for creating dynamic, real-time, and on demand smart medical devices specific to need.

In one embodiment, the release of a storage depot's contents can be selective in nature, allowing for individual smart devices and/or contents within a device depot to be released on an as-needed basis. In simplest terms, the analogy would be that of a parking garage containing numerous vehicles within a multi-tiered storage area. When one or multiple cars are needed by their owners, the parking attendant can retrieve the specific vehicle/s of interest and deposit them at a designated location for pick-up.

In one embodiment, in the case of a smart device storage depot, the individual smart devices and/or components can be retrieved from their storage compartment and transported to the desired location (similarly to how on a smaller scale, a smart device 206 or components can be retrieved from compartment 210). If the smart device and/or components have autonomous propulsion/navigation capabilities, they can self-navigate to the corresponding anatomic location of interest. Alternatively, if the smart devices and/or components do not possess self-navigation capability, they can be transported to the anatomic location of interest by a transport device, analogous to a tow truck or flatbed.

### E. Changing Smart Device Form and Functionality: Ex Vivo Smart Device Depots

Although the present invention includes in vivo smart devices and their ability to be transformed in form and/or function using readily available in vivo sources of smart devices and/or components, another embodiment exists for creating pluripotent in vivo smart medical devices through the creation of ex vivo (i.e., external) smart device storage depots (see FIG. 5 for one exemplary embodiment). These ex-vivo storage depots would function in a similar manner to in vivo smart device depots (e.g., depot 210) but instead, reside outside of the host subject.

In one embodiment, ex vivo smart device depots offer a number of benefits which may not be readily available with their in vivo counterparts. Whereas an in vivo smart device storage depot would be somewhat limited in size and configuration (due to the anatomic locations in which they reside), ex vivo storage depots need not have similar size restrictions. One could in theory create an ex vivo storage depot in size and configuration similar to a backpack, which can be portable and carried by the host subject. Alternatively, an ex vivo storage depot could be created and worn in a manner similar to a smart watch 240 (i.e., wearable smart device storage depot), which would become a semi-permanent accessory to the host subject and always readily accessible, with little thought or effort with regards to its presence.

In one embodiment, another ex vivo storage depot option could take the form of an auto-injector 500 (see FIG. 5 which shows the storage compartments 501-508 and injector portion 530), which would be analogous to an Epipen®, which is currently used for emergent administration of epinephrine for anaphylactic shock. But instead of solely housing a pharmaceutical, the ex vivo storage depot 500 would contain a variety of smart devices 512-517 and/or components (i.e., biospheres, nanobots, tools (cutting, scraping, attachment, etc.), liquids, etc.), which once properly sanitized and injected could be used to construct new smart devices and/or reconfigure or transform existing in vivo smart devices.

In one exemplary embodiment, a host subject with a history of heart disease experienced an allergic reaction, which resulted in anaphylactic shock and heart failure. Upon administration of a conventional Epipen®, the allergic reaction would be addressed but not any underlying cardiac related complications, such as heart failure, coronary artery ischemia, or cardiac arrythmia. In the exemplary embodiment, the present invention would replace the Epipen® with a smart device storage depot 500 which contains, for example, smart drug reservoirs 518 containing epinephrine, smart injection needles 519, smart coronary artery stents 516, nanobots 509, biospheres 513, assay smart devices 517, and the deconstructed components of a smart cardiac pacemaker 514, 515. A compartment ejector system 509 for each compartment 501-508 pushes the component such that, for example, the assay smart device 517 passes through opening 510 into a common tube 521, where the auto-injector system 511 pushes the smart device 517 through opening 520 and to the body injection system (not shown).

In one embodiment, in addition to the administration of epinephrine, the smart devices/components contained within the ex vivo storage depot 500 could contain devices and/or drugs to treat a variety of concomitant cardiac complications.

In one embodiment of the present invention, unlike an existing Epipen® which includes a single chamber, an ex vivo storage depot 500, for example, could be constructed to contain multiple chambers 501-508, each of which contains a variety of smart devices and/or components specific to a given pathology, anatomic region, diagnostic action, or therapeutic intervention

In another embodiment, individual smart devices or components could be individually packaged or compartmentalized within the storage depot (for example, injector system 500), allowing for the devices/components to be discharged on an individual basis (see FIG. 5). This would provide maximum flexibility in smart device and/or component delivery, while maintaining the integrity and availability of all other smart devices and/or components contained within the storage depot.

Another benefit of an ex vivo storage depot is the ability to replace, restock, and reconfigure its internal contents and/or structure. The portability and easy accessibility of an ex vivo storage depot provides for ease of use and modification of internal contents to a far greater degree than in vivo storage depots. The smart medical devices and/or components contained within an ex vivo storage depot can be updated and refined by the user whenever a given host subject's medical profile was to change, which could be in accordance with changes in real-time data, alteration in disease diagnosis and/or treatment, or risk adjustment.

In one exemplary embodiment, a host subject is planning a long trip in which he/she will be away from their normal healthcare providers for four weeks. While biodata will continue to be collected and analyzed by the program 244 in real-time, and existing in vivo smart devices and components will continue to operate in an unimpeded fashion, a number of preventative smart devices and/or components may be deemed necessary by the program 244 and/or user in the event of an unexpected medical emergency. Some of these potential medical emergencies may be specific to the geographic location in which the host will be traveling or the activities they may be engaged in.

In order to prepare for these potential adverse events, in one embodiment, a number of smart devices and/or components are identified by the program 244 and/or user, which are currently not contained within the host subject or existing storage depots (both in vivo and ex vivo). By having the ability to modify existing storage device contents or expand storage device capacity, the smart device inventory and functionality can be easily modified by the program 244 in keeping with the host subject's changing medical conditions or potential needs.

In one embodiment, the creation of multi-compartmental ex vivo storage depots (see FIG. 5, for example) is another unique feature of the present invention, for it provides an easy-to-use methodology for smart device and/or component inventory, replenishment, replacement, or utilization. In one embodiment, compartments can be created based upon a number of variables including (but not limited to) historical smart device usage, host medical profile, individual disease states, environmental or occupational exposures, or pharmacology.

In one embodiment, the implementation of an ex vivo storage device could be automated or manual. In an automated model, the program 244, using AI, would serve as the selection and triggering mechanism for the selection of the specific smart devices and/or components. In manual delivery operation, an authorized end-user would serve as the triggering mechanism after identification of the appropriate smart devices and compartment by the program 244. The input and decision-making process could be provided by either AI or human intelligence.

In one embodiment, in the event that the host subject is the party responsible for manual triggering of a multi-compartmental ex vivo storage device, visual aids could assist in the selection process. As an example, the triggering mechanism could be color coded to assist the user in identifying the specific compartment or context-specific smart devices and/or components of interest.

In one exemplary embodiment, compartmentalized cardiovascular smart devices would be designated by the color red. In the event that the host subject was experiencing a medical issue related to the cardiovascular system, a prompt by the program 244 after its analysis thereof, could alert the host to trigger the “red” compartment (i.e., light 522 in FIG. 5, for example).

In one embodiment, when compartments are specialized, a secondary prompt (e.g., numerical code) by the program 244 could be included, such as “Red-3”. For safety and security purposes, a second remote authentication (e.g., authorized cardiologist) may be required by the program 244 before the triggering mechanism is activated by the user/program.

Ex vivo storage depots serve an important function by expanding the inventory and diversity of readily available smart medical devices and/or components, beyond what is available in vivo. The addition of ex vivo storage device depots could dramatically expand the storage capabilities of smart devices while also providing for smart device functionality which might not be readily available through in vivo devices alone.

In an exemplary embodiment, if a completely unexpected pathology was to occur, which was not anticipated based upon the host subject medical profile, such as the host subject being exposed to anthrax (which can be effectively treated with antibiotics, including for example, Ciprofloxacin® or Vibramycin®), these antibiotics may not be readily available within existing in vivo smart devices or device depots, but could be readily accessible in an ex vivo storage depot.

In the exemplary embodiment, once the diagnosis is established through real-time biodata collection by the user and/or smart devices' program (i.e., programs 244, 234, etc.), and analysis is performed by the program, the host subject is provided by the program with the diagnosis and recommended treatment thereafter. Since the host subject's medical data is all combined in a single all-inclusive database 233 (or other external database), the recommended treatment is cross-referenced by the program 244 with the smart device database (i.e., memory 232), and the requisite smart device (i.e., smart device 206, which in this case would house antibiotics in compartment 210) is located within an ex vivo device storage depot (i.e., storage depot 500), along with the specific compartment (i.e., compartment 503) in which it is housed.

Through wireless transmission, the host subject is alerted as to the storage depot (i.e., storage depot 500) and its compartment (i.e., compartment 503) for injection. In addition, in one embodiment, a visual cue is created by the program, by lighting the ex vivo storage device (i.e., light 522) and specific compartment (i.e., compartment 503) in which the appropriate drug reservoir is located.

In the exemplary embodiment, the host subject removes the corresponding device depot (i.e., depot 500) and selects the specific compartment (i.e., compartment 503) in which the antibiotic drug reservoir (i.e., storage compartment 210 of smart device 206) is stored. (In another embodiment, the antibiotic could be held in a compartment similar to compartment 508 which held epinephrine 518 in a previous example.)

In the exemplary embodiment, a confirmation signal is received from the user and/or program 244, verifying the order, and the auto-injectable trigger (i.e., trigger 511) is activated, releasing the smart device with drug (i.e., smart device 206 with compartment 210), or the drug (i.e., antibiotic 518) itself from the appropriate compartment (i.e., compartment 503 or 509, respectively), via exit 520, which device 206 or drug 518 is then injected into the host subject.

In the exemplary embodiment, through a series of wireless transmissions and security checks by the program regarding the process above, the antibiotic drug reservoir (i.e., from compartment 508) and/or corresponding in vivo smart device with drug reservoir (i.e., from compartment 503), are released into the host subject bloodstream where the drug is injected. Continuous host biodata is collected by the smart device (i.e., device 206) and/or other medical equipment (i.e., biospheres, nanobots, microbots, catheters, pacemakers, etc.), and analyzed by the program 244, to assess host response to therapy and to determine whether additional intervention is required.

In the exemplary embodiment, if the treatment was to require the injected smart devices and/or components/drugs to navigate to a specific anatomic location to perform the required action, they would do so through either independent or assisted navigation, as described above and in the incorporated patents/applications.

The above exemplary embodiment demonstrates how an ex vivo storage depot can be accessed and used to deliver the required smart device and/or components/drugs in response to an immediate medical problem. However, in one embodiment, in a case where the host subject is incapable of accessing the required ex vivo device storage depot due to mental of physical limitations (e.g., unconscious state), then the presence of a portable and easily accessible ex vivo storage depot would have limited value, unless an alternative injection option could be found.

In one embodiment, the present invention would encompass an authorized third party who can administer the appropriate ex vivo storage depot/compartment. This could be accomplished given the appropriate security and safety protocols, which will be discussed below.

In another embodiment, a robotic ex vivo storage depot would include a robotic component (i.e., a device similar to an automatic drug delivery system attached to the host body) which would become the delivery mechanism for introducing the storage depot 500 into the host subject. In this embodiment, the robotic device (not shown) can be activated in an automated fashion by the program 244 if and when a medical emergency takes place, as defined by real-time biodata collection and analysis by the program 244. In a similar fashion to the program 244 notifying the conscious host subject as to the medical emergency and required intervention, the robotic injection device would become activated if the host subject fails to or is unable to respond to emergency alerts. Once again, strict security protocols are required by the program 244 in order to ensure that the smart device delivery is required, appropriate, and directed to the correctly identified host subject.

In another embodiment, in the case of a medical emergency in which the required smart medical device and/or components/drugs are not readily available, an alternative option is to deliver the requisite smart device depot 500 to the host subject. One way to do this is with the use of drone technology, which can identify and track the location of the host subject for delivery. Since smart in vivo devices possess the ability to continuously transmit real-time host specific data through wireless transmission (i.e., transmitter/receiver 218, transmitter 206, receiver 209) within both local and wide area networks, the presence of a medical emergency could be readily identified and tracked by the program 244. As described in the incorporated patents/applications, the identity and location of the host subject could be readily identified by the program 244 based upon the unique transmission of in vivo smart devices.

In one embodiment, once the emergent data is received and analyzed by the program 244, communications with the host subject and their pertinent in vivo smart medical devices would automatically take place by the program 244, in an attempt to diagnose and intervene. If this communication was unsuccessful and/or the in vivo smart devices are not capable of satisfactorily addressing the medical emergency as determined by the program 244, then contingency plans are automatically activated by the program 244. In addition to the program 244 alerting emergency medical personnel and authorized healthcare providers, an automated delivery mechanism (e.g., drone, autonomous vehicle, etc.) could be triggered by the program to transport the requisite smart devices and/or components/drugs to the host location.

In one embodiment, upon arrival, the robotic component of the smart device delivery system can be activated by the program 244, once the host subject identity has been authenticated and verified by the program 244. The corresponding smart medical devices and/or components/drugs would then be introduced into the host subject and continuous real-time data collection and analysis by the program 244 would take place to measure the clinical response, as well as to determine any additional intervention required. Simultaneously, continuous medical updates would be provided by the program 244 by electronic communication methods (i.e., email, fax, text, etc.), to emergency medical personnel as they travel to the host location for additional medical care delivery.

The net result of the present invention is that smart medical devices and/or components/drugs can be introduced to the host in a variety of manners and locations, irrespective of the host subject's ability to provide assistance. The technology of the present invention is designed to function in an autonomous or semi-autonomous fashion, in accordance with available resources.

### F. Artificial Intelligence in Pluripotent Smart Medical Devices

A number of artificial intelligence (AI) features have been described in the incorporated patents/applications, which are referenceable to the present invention. At the core of smart medical device AI is machine learning and the wide array of neural networks which have been derived and are continually evolving over time.

Machine learning can function in a variety of ways, all of which are directly applicable to the invention. These include the following:
1. Descriptive: use data to explain what has happened (i.e., past events).
2. Predictive: use data to predict future events.
3. Prescriptive: use data to make suggestions as to what action should be taken.

In one embodiment, AI relates to the present invention in the descriptive and predictive data-driven deliverables, which can be used for medical diagnosis, while the predictive deliverables can be used for optimizing medical intervention and treatment.

In one embodiment of the present invention, there is continuous creation, recording, storage, and analysis by the program 244 of host-specific real-time dynamic medical data, which provides the foundation for real-time diagnosis and treatment. This context and user-specific data can in turn be correlated by the program 244 with external data derived from millions of other hosts, thereby providing a rich data source for identifying pertinent data trends and confirmed clinical outcomes.

Neural networks represent a class of machine learning algorithms which are modelled on the human brain. In one embodiment, a number of neural networks are encompassed by the present invention including (but not limited to): deep neural networks, convolutional neural networks, recurrent neural networks, generative adversarial networks, and transformer neural networks. As neural networks continue to evolve, they will inevitably improve in performance and provide the basis of the computationally intensive and time sensitive tasks of the present invention.

Deep learning represents multi-layered neural networks which can process vast amounts of data to determine the relative weight of each link in the network. As a result, they provide valuable insight as to the relative importance of each individual data element. They are currently used in a wide array of applications including (but not limited to) autonomous vehicles, chatbots, and medical diagnostics. The greater the number of layers contained within a given neural network, the greater the potential to process complex data and relationships.

In one exemplary embodiment, if analysis by the program 244 of real-time host smart medical device biodata identifies a high statistical probability for an acute brain infarct, additional data is directed by the program 244 for confirmation and/or revision of the suspicion. If continuous data collection and analysis by the program 244 does indeed confirm the suspected diagnosis, the program's 244 AI can be used to direct both existing smart medical devices and the creation of new smart medical devices and/or components towards definitive establishment of the diagnosis, and further characterizing it through the collection and analysis of more detailed biodata related to anatomic location, severity, and related clinical findings.

In one embodiment, in addition to this refined diagnosis, AI can also be used in determining the optimal intervention (i.e., treatment), based upon a number of factors, including (but not limited to) available resources (including in vivo smart medical devices and components), host medical profile, host specific anatomy, and established population-based treatment options.

In one embodiment, in the case of effecting optimal intervention, the treatment of choice by the program 244 includes local infusion of thrombolytics directly at the site of vascular occlusion. If this cannot be readily achieved through existing in vivo smart medical devices, then the program can query the database (i.e., memory 233 and/or 234) of accessible smart medical devices and/or components to determine whether the required smart medical devices and/or components can be assembled and/or transformed and transported to the anatomic location of interest. In this manner, AI plays the role of data aggregator and analyzer, for both medical diagnosis and treatment. In the course of performing these duties, the program's 244 AI can direct the identification, assembly, transport, and function of in vivo smart medical devices towards the required actions required.

As it relates to new and evolving AI, transformer neural networks currently represent the state of the art, based upon their ability to solve sequence-to-sequence tasks while also handling long range dependencies. A transformer is a type of deep learning model which differentially weighs the significance of each individual data element, along with its relationship to other data. Transformer models apply evolving mathematical techniques (e.g., self-attention) to detect subtle ways various data elements influence and depend on one another. A transformer model learns context and meaning by tracking relationships in sequential data and as a result, is another feature of the present invention. Thus, in one embodiment, the program's 244 AI can include a transformer model in its analysis of the data collected by the smart devices.

Regardless of the specific form of AI in use, in one embodiment, program AI serves an important role in the present invention due to the large quantity of real-time data being continuously generated by the smart medical devices, as well as the importance in correctly analyzing the data for immediate action. While the present invention also provides an avenue for external human-derived data analysis and intervention, the preferable route is one in which the smart medical devices and/or components function in an autonomous fashion.

### G. Security Features for Pluripotent Smart Medical Devices

A number of individual safety and security features for smart medical devices have been previously discussed in the incorporated patents/applications. These covered a variety of related topics which included the following:

### 1. Smart Device Authentication, Verification, and Authorization.

As is the case in any computer-based system, in one embodiment, a number of authentication technologies are readily available to verify the identity of the operator (whether human or computer) and ensure that they are properly vetted to access the system and input directives. Existing technologies which can be integrated into the smart medical device network include (but are not limited to) password protection, multi-factor authentication, token-based authentication, certificate-based authentication, out of band authentication, transaction authentication, and biometrics.

### 2. Software Updates and Modifications.

Remote access by authorized computer systems provides the ability to upgrade software on an as-needed basis. This provides a relatively easy method for fixes to software malfunctions, addition of new security and safety features, and expansion of functionality. In one embodiment, since all computer-based access is automatically recorded in a database (i.e., database 215), a cloud-based permanent record is readily available for routine or emergent audits, in keeping with program security and safety guidelines.

In one embodiment, software upgrades and modifications are important to all in vivo smart devices, regardless of their specific function and technical components. In the event that some smart devices do not possess compatible software, this may hinder inter-device communication and functionality. In one embodiment, in addition to upgrades through wireless transmission, physical add-ons of microcomputers may be required, which can take place through transporting smart devices, which may serve to implant the new and more sophisticated microcomputers into a designated port on the smart device.

In one embodiment, each individual miniaturized device (i.e., biosensor 221) which is embedded within the smart device (i.e., device 230) may contain its own internal computer and operating system (not shown, but similar in components to that of device 230), which has the capability of internal upgrades separate from the primary computer system (i.e., computer system 227) of the larger all-inclusive smart device (i.e., device 230). In one embodiment, this provides a method for numerous miniaturized components in different smart devices to operate synergistically with one another.

In an exemplary embodiment, suppose a specific type of biosensor (i.e., biosensor 100, 200, 201) requires an upgrade to enhance detection of a specific biochemical compound. These specific types of biosensors may be contained within a variety of different smart devices (i.e., smart device 206). By having the ability to upgrade software for each individual biosensor, the entire class of biosensors can function in concert with one another, irrespective of the specific type of medical device in which they are embedded.

In one embodiment, when a security threat is encountered, the individual operating systems (i.e., computer system 227) of the affected smart devices and/or subcomponents can be remotely shut down by the program 244. If and when the threat is aborted and/or nullified by the program 244, the operating system 227 can be remotely turned back on by the program 244 (externally, from computer system 214, for example), restoring function. This provides a method for containing and limiting the spread of security threats, while maintaining function of the other non-threatened components within an individual smart device.

### 3. Smart Device Quality Assurance (QA) and Quality Control (QC).

In one embodiment, routine testing is required for all smart medical devices and their individual subcomponents in order to ensure they are operational, accurate, secure, and safe. In addition to the QC testing of each technical component, routine testing is also performed on the communication systems (i.e., transmitter/receiver 218, transmitter 208, receiver 209, etc.), which are also important to device operation and inter-device coordination.

In one embodiment, all QA and QC testing results can be automatically recorded by the program 244 in a QA/QC database (i.e., memory storage 233 or OTHER external database) for review and analysis by the program 244. Whenever the program 244 performing routine testing identifies a potential deficiency, an automated escalation pathway can be triggered by the program 244, which ensures that the smart device (i.e., device 206, 230, 300) and/or subcomponents of concern are removed from routine operation, until the deficiency in question has been satisfactorily addressed. In some circumstances where simple shut down by the program 244 of the involved component and/or device is insufficient, extraction of the smart device may be required in order to ensure that the host patient and/or other smart devices are not adversely affected.

In one embodiment, the present invention, which may be applicable to the QA/QC testing and repair process, is the ability to repair and/or replace the involved subcomponents or components of the smart device. In one embodiment, designated repair smart devices may be dispatched by the program 244 to the location of the smart device in disrepair, where they can replace and/or repair the deficient component in question (see the incorporated patents/applications for further explication). Once the repair or replacement has been completed, remote QA/QC testing can be performed by the program 244 to assess whether the operation has been successful. If the program 244 determines that it has, the smart device and/or subcomponent of concern can be recommissioned and restored by the program 244 to active duty. If unsuccessful, the component and/or device will remain out of commission and/or extracted if necessary by various methods (see the incorporated patents/applications for various methods).

### 4. Break Glass Feature.

In one embodiment, the “break glass” feature of the present invention is primarily designed to serve as a safeguard for an emergent high security situation, which could entail severe injury or death, if left unattended. Under normal circumstances, in one embodiment, a well-defined security protocol establishes chain of command for intervention. In this chain of command security protocol of the present invention, a well-defined hierarchy is established by the program 244 to define what parties have the power to intervene in smart medical device actions. A number of variables are defined by the program 244 including (but not limited to) the specific type of smart device, its location, the clinical context in which it operates, the target destination, other smart devices in which it interacts, and the scope of operation.

While the conventional security and safety protocol of the present invention is designed to address most issues of concern, the possibility of a life-threatening emergency requiring immediate action may occur, which is so time sensitive that the delay associated with routine security protocols would prove to be costly. In such a truly emergent situation, the break glass feature of the present invention provides a mechanism for immediate intervention. However, the user would have to ensure that when circumventing the security protocols in place to utilize this feature, the system program 244 is not exposed to malicious activity.

In one exemplary embodiment, a patient with an acute stroke due to acute occlusive thrombus in the middle cerebral artery is being treated through a multi-smart device intervention. In this planned intervention, one smart device is designated to locally infuse a thrombolytic agent, a second device is designated to follow with a drilling device, while a third device deploys an umbrella (e.g., device 403) to trap any small thrombus fragments. The collective action of the three devices aims to reduce the thrombus burden, so as to restore blood flow to the area of acute infarction before irreversible neural injury occurs.

In this exemplary embodiment, in order to work properly the drilling and umbrella devices (see FIG. 5, for example) must act in a coordinated fashion, so that no thrombus fragments are allowed to pass into distal vessels and cause downstream occlusion. However, in this exemplary embodiment, if the drilling device fails to recognize the lack of responsiveness on the part of a faulty umbrella smart device and begins its drilling operation, this will effectively result in numerous small thrombus fragments being released into the bloodstream, which would inevitably lodge in distal cerebral artery branches and cause multiple new brain infarcts.

In such an exemplary scenario, the only way to prevent a catastrophe would be for the program 244 to emergently turn off the drilling smart device. This scenario is exemplary of the “break glass” feature, which allows emergent action.

In one embodiment, in the event that a high-level safety or security risk is identified by the program 244, authorized parties can intervene to avert the crisis at hand, with multiple parties required to confirm the necessity of such intervention (in an escalation pathway as described previously). By the program 244 having multiple parties sign off, this theoretically averts inappropriate action on a single operator's part.

Thus, in one embodiment, if and when the break glass feature is deployed by the program 244, a series of urgent alerts would automatically be transmitted by the program 244 (by electronic means like text, fax, email, etc.) to all parties with the corresponding high security clearance, notifying them of a potential security breach. In turn, any and all of those parties notified, would have the ability to be rapidly authenticated by the program 244 and given access to the smart devices in question. Once established, these individuals would have the capability of overriding or modifying the break glass command, as clinically indicated.

In one embodiment, as is the case with all other data inputs, the corresponding data is automatically recorded by the program 244 in the operational database (i.e., storage 233) and amendable to both computerized and human audits and analyses.

### 5. Hierarchical Privileges.

In one embodiment, regardless of whether data input or output involves humans or computers, the sharing of data/information requires a delineation of privileges, in association with both identity and context. Just as is the case with the clinical practice of medicine, in one embodiment, privileges to sensitive data must be narrowly defined, in accordance with the profile of the involved parties. This principle holds true for the ability to access and input data relating to smart medical devices of the present invention.

In one exemplary embodiment of a semi-autonomous smart medical device, a human operator may be tasked with supervising and assisting with smart device navigation. But in order to ensure that the operator has the appropriate training and clearance, each individual interacting with smart devices must first be properly vetted and assigned specific privileges in accordance with what data they are privy to and what data and associated actions they can input.

In one embodiment, a number of variables will be considered in the definition of these privileges including (but not limited to) the specific type of smart medical device, the clinical context in which it is being used, the anatomic location in which it travels, other medical devices in which it interacts with, the identity of the host patient, and the subcomponents contained within the smart device. In some instances, privileges for an authorized operator may be individualized for some subcomponents within a given smart device and not others.

In an exemplary embodiment, a smart medical device with an embedded miniaturized surgical instrument may have privileges assigned to a surgeon but not a cardiologist, whereas an embedded electrophysiologic sensor within the same device may have assigned privileges to a cardiologist, but not a surgeon. At the same time, in one embodiment, the navigational system within the same smart device may have privileges assigned to a biochemical engineer, who does not possess similar privileges to the surgical or electrical biosensor. In this manner, privileges related to a single smart medical device may be assigned to individual subcomponents or operating systems at the exclusion of others. A select few, may have more expansive or even complete privileges for a smart medical device (i.e., smart device “super-user”), and these select few are often the ones with the highest security clearance allowing for the break glass application.

In a similar manner, in one embodiment, hierarchical privileges and associated data accessibility can also be assigned to other medical devices and/or computers. This defines how other smart medical devices and/or their subcomponents may function in tandem with other smart medical devices. In one embodiment, as previously cited in the use case, multiple smart devices working in concert with one another were assigned to the task of removing occlusive thrombus from an occluded cerebral artery. One smart device was responsible for drilling thrombus, another with infusing a thrombolytic agent, and another deploying an umbrella to trap small thrombus fragments. The ability for these individual smart devices and their subcomponents to interact and communicate with one another is in part defined by their privileges, which define communication protocols and data accessibility between the computers within each individual smart medical device.

In one embodiment, these privileges may be of variable duration so as to allow an authorized end-user a defined time period in which data is accessible. This serves as a security feature limiting both the time and extent to which a given human or computer may have access to a given smart medical device.

In one embodiment, all interactions between authorized end-users (both human and computers) and smart devices can be recorded by the program 244 into a database (i.e., database 233) for analysis. In the event that a given interaction was deemed to be improper (i.e., safety and/or security risk), by a user or the program 244, the associated privileges can be modified (e.g., downgraded or terminated) based on the determined level of negative interaction by a team of clinical and technical experts.

In one embodiment, another security and safety feature which can be incorporated into the present invention is blockchain technology, which can lead to the creation of a virtual secure ledger for assignation and determination of smart medical device privileges, which cannot be readily altered. The incorporation of blockchain technology in the present invention provides a shared immutable ledger that facilitates program recording and tracking of data transactions and communications within the diverse smart medical device network.

### 6. On/Off Functionality.

In one embodiment, since smart medical device safety and security is in part related to the ability to shut down function on demand, on/off functionality is required at both the level of the entire smart device and its subcomponents. In one embodiment, under some circumstances, on/off functionality may also be applied to multiple smart medical devices, when working in concert with one another, as illustrated in the use case example cited above, of the occluded cerebral artery. In that example, malfunction on the part of the umbrella device (which serves to trap small thrombus fragments), will result in simultaneous shut down of both the umbrella and drilling devices, which are dependent upon one another for safe operation.

In one embodiment, on/off group functionality is particularly relevant to nanobots and microbots (which will be subsequently referred to as “bots”) (e.g., device 206, 230), which are often present in large numbers given their small size (see the incorporated patents/applications). These are in effect, also smart medical devices, but in miniaturized size. As a result, in one embodiment, in vivo smart bots often function in groups, which can be collectively communicated with through the transmission and receipt of a unique signal frequency. In the event that a group of bots tasked with a specific task or function requires termination of the task in question, a single command by an authorized end-user can trigger the “off” function contained within each bot, thereby immobilizing an entire group of bots, which can number in the hundreds, thousands, or even millions.

In one embodiment, this on/off functionality may also serve as an important feature in smart medical device quality control (QC). When routine testing is performed by the program (e.g., programs 244, 232) on the various subcomponents contained within a given smart device (e.g., device 206, 230, 300), the program may determine that one or more of these components (e.g., sensor 221) (as well as the entire device itself) is no longer properly functioning. In such a scenario, the “off” function may be activated by the program, rendering the individual subcomponent/s or device no longer active. If and when the subcomponent and/or device functionality is returned, the “on” function can be activated by the program, so that function is restored. Return of function can be as simple as recalibration or as extreme as replacement of the component in question.

In one embodiment, on/off functionality can also be used in the setting of preventative maintenance, where various components of a given smart device may require calibration, software updates, or repair. During the time period in which preventative maintenance is performed, the associated smart device and/or components are deactivated (i.e., turned off) by the program 244, and subsequently returned to action by the program 244 once the maintenance is successfully completed. All actions taken from the time of on/off activation can be recorded in a database (e.g., database 233, 232) by the program 244 and audited by the program 244 for safety and security purposes.

In one embodiment, the previously described “break glass” function of the present invention represents an extreme case in which “off” functionality is activated in an emergency situation by the program 244. Once activated, as noted above, a series of safety and security measures would be required by the program 244 before reactivating the device.

In one embodiment, on/off functionality need not be exclusively binary, but instead can be scalable. In an exemplary embodiment, a smart vascular catheter (e.g., catheter 300) has been successfully positioned at its destination site within the superior vena cava, thus, the navigation component of the device may no longer require complete activation by the program 244, but instead can be placed in a semi-active mode by the program 244. In one embodiment, using a scale of 0-9 for on/off functionality, where 0 is completely off and 9 is completely on, the navigation component (e.g., navigation system 305) of the smart device while in proper position, may now be adjusted to 3 by the program 244. This allows for energy conservation while also minimizing the degree of sensitivity, with regards to repositioning,

If, in this exemplary embodiment, the device's position was to deviate by more than five (5) cm as determined by the program 244, the navigation system (e.g., 305) would be triggered by the program 244 to readjust device positioning. On the other hand, if the on/off functionality was set to a higher level of 6 (as opposed to 3), the positioning triggering mechanism of the program 244 may be activated at a positional change of three (3) cm (as opposed to five (5) cm). Thus, the on/off functionality of the present invention can be scalable on an as-needed basis.

In one embodiment, the activation or modification of the on/off functionality can be controlled by a variety of authorized sources, including (but not limited to) the program 234 of a smart device (e.g., device 206) in question, another smart device (e.g., device 230), external computer (e.g., system 214), or a human operator—by exceeding a predefined threshold.

In one embodiment, on/off functionality can also be controlled by a timer set by the program 244 for a variable duration, in a manner analogous to a home smart device controlling lighting). In addition, on/off functionality can be automatically triggered by the program 244 by changes in the health status of the host patient.

In an exemplary embodiment, biosensors (e.g., biosensors 201), embedded in a number of smart devices, serve to measure cytokines in the bloodstream. Since these have remained non-measurable over a prolonged time period, the corresponding biosensors have been effectively turned off. However, if the host patient's health status was to suddenly change and a fever was detected by the program 244 or a user, the corresponding cytokine biosensors could be automatically turned back on by the program 244, and they would now become reactivated. This illustrates the dynamic nature of smart device on/off functionality, which can be modified by both manual or automated means.

### 7. Inter-Device Communication.

In one embodiment, since inter-network communication is important to smart device performance, the program (e.g., program(s) 244, 234) records and analyzes all communications which occur at or between smart medical devices and their subcomponents. Since each device and its subcomponents have their own unique signal profile, the source and identity of all communications can be readily identified by the program(s), with a number of recorded communication metrics including (but not limited to) identity of the device, its location in the host, nature of the communication, its duration, time, frequency, and subsequent actions taken. In the event that a communication of concern was identified, the device/s in question can be proactively monitored by the program and intervention take place by the program and/or user when indicated.

In one embodiment, the lack of communication, in some situations, may also serve as a point of concern. Continuing the exemplary embodiment noted above with respect to the acute occlusive thrombus, a multi-device action is planned by the program(s) where multiple smart devices are acting in concert with one another in order to facilitate a complex action. In this exemplary embodiment, the middle cerebral artery is being treated through a multi-smart device intervention. In this planned intervention by the program(s), one smart device is designated to locally infuse a thrombolytic agent, a second device is designated to follow with a drilling device, while a third device deploys an umbrella to trap any small thrombus fragments. The collective action of the three devices by the program(s) aims to reduce the thrombus burden, so as to restore blood flow to the area of acute infarction before irreversible neural injury occurs.

In one embodiment, in order to work properly it is important that the drilling and umbrella devices act in a coordinated fashion as implemented by the program(s), so that no thrombus fragments are allowed to pass into distal vessels and cause downstream occlusion. As a result, the communication between these two devices is important to ensure clinical success and avoid an iatrogenic adverse outcome.

In this exemplary embodiment, the communication initiated by the drilling device is not verified by the program(s) and responded to by the umbrella. Second and third communication attempts by the program(s) also fail, resulting in cancellation of the proposed action by the program(s). The infusion device proceeds as planned as determined by the program(s), since it does not require assistance from a second device. The umbrella device is recalled by the program(s) and a new device is dispatched by the program(s). Once this has been completed and the communication signals are transmitted and verified by the program(s), the operation can recommence.

In one embodiment, in addition to having control over its own navigational system (e.g., tail 260, flaps 261) and the ability for multifunctional autonomous operation, individual smart medical devices also possess the ability to communicate and directly influence operation of other smart medical devices, resulting in group coordinated activity. Using AI and machine learning in their programs (e.g., program 234), these devices can actively learn and adapt to one another's navigation, particularly when the activities they engage with one another in are repetitive in nature. By using each individual smart device's ability to send and receive signals, the programs of the smart devices can actively track one another's four-dimensional (4D) in vivo location and directional movements. In the event that a smart device's movement is perceived to be contrary to expectations by the program, a warning signal can be transmitted by the program-via electronic methods as stated previously—for enhanced and continuous evaluation of the device in question. This serves as an added safety and security measure in the event of smart device malfunction or malevolent manipulation.

In one embodiment, all inter-device communications can be recorded by the program 244, 234 into a centralized database (i.e., database 233) for analysis, creating an added security/safety feature, along with data to drive future software development and technology refinement.

In one exemplary embodiment, to illustrate how inter-device communication and coordination can work, a patient with lower extremity deep venous thrombosis (DVT) and saddle pulmonary emboli (PE) will have two separate procedures undertaken in a clinical setting to address the lower extremity and pulmonary arterial thrombi. For treatment of the lower extremity, an inferior vena cava (IVC) filter will be deployed by the program, from a single smart medical device, which acts to trap migrating thrombi originating from the lower extremity DVT.

In this exemplary embodiment, for treatment of the PE, a combination of smart medical devices will be utilized. These include a device infusing a local thrombolytic agent to help dissolve the occluding embolus, which is later followed by a pair of smart devices acting in coordination of one another. The first smart device of the pair will deploy a mechanical drill for breaking apart the remaining thrombus (see incorporated patents/applications), while the second device will deploy an umbrella device a few centimeters away from the drilling device, which serves to catch small embolic fragments and preventing them from travelling downstream, where they could obstruct distal pulmonary arterial branches. It is important that these two devices operate in a synchronous fashion to one another (by program rules), to avoid iatrogenic complications.

Thus, in this exemplary embodiment, this collective operation involves four separate smart medical devices, two of which will act independently on their own programs, and two which must act in a well-orchestrated and coordinated fashion from their own programs or from external programming. For the latter two which act in concert with one another, inter-device communication is important to achieve a successful clinical outcome. For the other two devices, inter-device communication is helpful, but the timing and importance of this communication is of a lesser degree.

In the first action taken in this exemplary embodiment, the smart device deploying the IVC filter (e.g., device 516; see FIG. 5 for examples of various smart devices in the compartments 501-508) is dispatched by the program 244, and once the program has successfully verified the filter's correct positioning, the program has the smart device release the IVC filter. Subsequently, the sensors contained within the IVC filter verify their individual positioning relative to the IVC walls and the program tests each individual strut for proper functionality. Once the program completes this internal test, the program has the smart device deliver a test injection of water-soluble particles to test the positioning and trapping function of the IVC filter. If the test is satisfactory per program analysis, the smart device returns to its baseline anatomic poisoning, using its autonomous navigation system.

In this exemplary embodiment, now that the lower extremity DVT has been satisfactorily addressed, the next (and clinically more important) task is to deal with the large centrally localized PE. Since this involves the deployment of three separate smart devices in a two-stage procedure, the timing and order of smart device deployment is important.

In the exemplary embodiment, the first device deployed is the device tasked with infusion of the thrombolytic agent, of which anatomic positioning is important to avoid systemic complications. For this reason, navigation and precise positioning of the infusion port in direct proximity of the thrombus is important for optimizing clinical outcome. The manner in which this targeted navigation takes place has been described in detail in the present description of the invention and in the incorporated patents/applications.

In this exemplary embodiment, after localized infusion of the thrombolytic agent has completed, the remaining thrombus can be physically removed through the coordinated efforts of smart medical devices containing a drilling device and umbrella device. In one embodiment, the drilling device (e.g., device 519) is tasked by the program with physical breakup and suctioning of the thrombus, while the umbrella device (e.g., device 515) is tasked by the program with catching all downstream debris which becomes detached from the primary thrombus and enters the bloodstream. If these debris fragments were not trapped by the umbrella device, they would pass into distal cerebral artery branches, occlude smaller vessels, and produce a series of infarcts. As a result, the coordinated efforts of the drilling device, suction apparatus, and umbrella by the program 244 are important to eradicate the thrombus and prevent strokes from occurring.

In the exemplary embodiment, while exact poisoning of each device and its subcomponents is important to operational success, of greater importance is the coordination and timing of the devices by the program, to ensure that each individual device and its subcomponents are synergistically functioning both independently and in concert with one another.

In one embodiment, the ability of individual smart devices and their subcomponents to communicate with one another provides an important tool for coordinated activities. As each individual device navigates to its intended anatomic position, it can send a series of signals (via transmitter 208, for example, which may be located on all smart devices) to update other devices (which are receiving signals via receiver 209, which may be located on all smart device) and computer systems (e.g., system 214) of its position. Once it has successfully arrived at its intended destination, it can signal the other devices, which in turn can communicate its position. Once all involved devices have reached their intended positions and communicated with one another, testing of the devices and subcomponents can be performed by the program(s) 244, 234, prior to commencement of the procedure.

In one embodiment, in the event that a given device or its subcomponents is/are not properly positioned or functioning, the program will not allow the procedure to proceed until the requisite problem has been resolved. In addition, if any device does not properly communicate with its designated partners as determined by the program(s), the procedure remains on hold. This inter-device communication provides an important safety feature to ensure that the coordinated actions of each device are verified before beginning the procedure.

In this exemplary embodiment, if the device containing the umbrella apparatus is not in the correct position, is not fully functional, or has failed to communicate with the drilling device, as determined by the program(s), in any of these scenarios, the drilling device will not begin until it has received communication from the umbrella device and/or the central computer (e.g., system 214) that all required steps have been verified and the program 244 can allow the procedure to proceed.

In an exemplary embodiment, one scenario could be that the drilling device violates the established protocol and begins operation without first receiving the required verification, which could in effect create detached thrombus fragments from travelling distally and obstructing smaller downstream vessels. However, in the present invention, a number of safeguards exist to prevent and/or limit the potential of such an adverse action. In one embodiment, these include (but are not limited by) the following:
1. Automated termination in absence of corroborating signal from partnering smart device.
2. Automated termination upon receipt of distress signal from partnering smart device.
3. Termination upon receipt of signal from central computer system monitoring communications.
4. Termination command from authorized human operator monitoring communications and smart device actions.
5. Activation of break glass feature causing immediate and irreversible shutdown.
6. Automated cessation of activity triggered by program's audit and analysis of inter-device communications.
7. Activation of intervention smart devices to minimize negative impact in the event of shutdown failure.

In one embodiment, the last item in which an intervention option is deployed by the program(s) could include a myriad of possibilities, in which specialized smart devices are deployed in an effort of damage control. In this specific exemplary embodiment, one option may include release of large numbers (i.e., thousands or millions) of specialized nanobots in the host patient, which possess the ability to release short distance and low frequency lasers into the nearby paths in which they travel.

In one embodiment, the lasers being emitted by these circulating nanobots are designed to ensure that they cause no damage to normal tissue they encounter but would serve to disintegrate any large particulate matter in their path. One of the applications of the present invention is the elimination of small thrombi and/or emboli which are freely circulating in the bloodstream, as in this specific case. By injecting large numbers, the circulating nanobots will effectively create a continuous stream of lasers as they pass through the area of clinical concern.

In one embodiment, under circumstances where the nanobots' location is restricted, a specialized injection site may be required in lieu of the normal introduction via the peripheral bloodstream. In this particular exemplary embodiment, where the blood/brain barrier may restrict nanobot entry into the cerebral arteries, an alternative intrathecal injection may be required. The net effect is that when an iatrogenic complication is identified by the program(s) related to smart medical device activity, other specialized smart medical devices may be deployed by the program(s) and/or user for treatment. Since every operation has the potential for an unplanned mishap, multifunctional smart medical devices can play a vital role in counteracting the negative impact. At the center of preventing such a mishap is inter-device communication.

### 8. Smart Device Elimination or Extraction.

In one embodiment, the present invention provides for an exit strategy in the implementation of smart medical devices, which includes either physiologic elimination from the host patient or physical extraction (see the incorporated patents/applications). In one embodiment, smart device removal from the host patient is normally an elective process, which may be triggered by a number of processes determined by the program(s) and/or user, including (but not limited to) smart device mechanical failure, completion of clinical task, smart device obsolescence, or requirement for smart device repair. In rare circumstances, the smart device retrieval may be the result of unexpected activity, and in such a case, an emergency evacuation is required, in order to alleviate any danger or adverse action.

In addition to the break glass option of the present invention, which has been previously described for immediate shutdown of a smart device under extreme circumstances, in one embodiment, an additional safety and security feature is a self-destruction option, in which an authorized operator can command the program to trigger an internal implosion device which causes the smart device to be destroyed, with minimal impact beyond the confines of the device.

In one embodiment, regardless of the mechanism of immobilization, a smart device which has been voluntarily or involuntarily decommissioned must have a mechanism in which it is removed from the host patient. The present invention provides for a number of elimination and extraction methods for this purpose.

In the present invention, the primary difference between extraction and elimination is that extraction is physically supported by another entity, while elimination does not require physical assistance. Elimination can be the result of a smart device passing into a physiologic system for removal (e.g., gastrointestinal tract, urinary system, respiratory system). In one embodiment, while nanobots are small enough to also be trans-dermally eliminated via perspiration, larger smart devices can also be limited trans-dermally through activation of a “boring” or tunneling device, which allows a puncture in the skin surface to be created in which they can exit the host patient.

In one embodiment, conversely, extraction requires physical assistance for device removal from the host. In one embodiment, this assistance can be provided from other smart devices or authorized human operators. In addition to extracting the smart device in its whole state, in one embodiment, smart devices may also have the ability to be broken down into subcomponents, depending upon the device structure and composition. In one embodiment, some devices may be constructed in an articulated format, allowing the individual articulated components to be detached from the central core of the device upon program command, allowing for extraction of multiple smaller parts. In another embodiment, other smart devices may have appendages (e.g., cardiac pacemaker), which provide a natural mechanism for disassembly prior to extraction. Lastly, in one embodiment, smart devices can also be physically downsized or fragmented through controlled implosion commanded by the program, rendering it into multiple smaller pieces for easier extraction. Regardless of the strategy employed, extraction includes the program implementing transport of the smart device in toto or in parts to a designated extraction site for final removal.

In one embodiment, the simplest method of extraction is via towing of one smart device by another. In one embodiment, in the event that a smart device is disassembled or broken into multiple pieces, multiple smart devices may be required for extraction. Alternatively, if a smart device is destroyed, resulting in multifocal debris and/or small components, in another embodiment, an alternative strategy can be utilized such as a vacuum or filter equipped smart device or lasers for complete dissolution of the smaller fragments. The smart devices participating in these extraction techniques may do so autonomously by program rules or under the direction of an authorized human operator.

In one embodiment, when physical extraction requires minor surgery, the smart device can be navigated by the program or towed to a designated superficial location and an incision made by an authorized operator or robot for final removal of the smart device. In one embodiment, when physiologic elimination occurs, the smart device in question can be captured by filtering the medium in which it passes (e.g., air, feces, urine).

In one embodiment, upon retrieval, the smart device and/or its subcomponents can be collected and subjected to additional testing on an as-needed basis. In one embodiment, biologic material is collected and can be retrieved from the storage device (i.e., storage depot 210, etc.) in which it was collected.

### 9. Anti-Hacking.

As is the case for any computerized system (and especially the case for in vivo medical devices), anti-hacking features are important to assure safety and security. In one embodiment, a number of technical solutions can be applied to the present invention including (but not limited to) encryption, blockchain, multi-party authentication, and biometrics.

In one embodiment, in the event that an unusual, unexpected, or unauthorized smart device action takes place, an automated alert would be triggered by the program which would notify authorized responsible parties via electronic methods (i.e., fax, email, text, etc.) for engagement and feedback. In addition, in one embodiment, the operator currently tied to the actions of the smart device in question would be required to undergo reauthentication and verification. In one embodiment, in the event that they failed to adequately provide this reauthentication and verification, and/or other authorized operators determine the action to be unsafe or contrary to the standard of care, then the program would immediately cancel the smart device from further action.

As previously stated, in one embodiment, other security features such as broken glass and/or smart device destruction can be deployed under extreme circumstances.

The present invention views smart autonomous medical devices not just as standalone objects, but in the context of the entirety of the computer-based network in which they reside and function. All of the computerized components create a series of nodes on a network, each of which serves as a point of potential compromise. In one embodiment, these include (but are not limited to) the smart device (i.e., smart device 230, 300, etc.) as a whole, the numerous embedded miniaturized devices (i.e., sensors 221) contained within each device, and the various externally located computers (i.e., computer system 214) which the smart device communicates with. In one embodiment, among these external computers are other smart medical devices, which routinely communicate and interact with the smart medical device of primary concern. In one embodiment, one additional node on the network is the human operator, who when properly authorized, may also play a role in smart medical device navigation and function. As a result, they also play a role in device safety and security. Thus, safeguarding safety and security must account for each and every one of these components.

While these individual safety and security features are also applicable to the current invention, an important topic of consideration is that of overall network security. In one embodiment, decision-making regarding the operability of in vivo smart devices can be performed by authorized human or computer end-users. In one embodiment, when human input is performed, all outside sources must go through an extensive verification and authentication process to ensure appropriate safety and security safeguards are maintained.

Traditional network security is based on a castle-and-moat concept, which makes it difficult to obtain access from outside the network, but once inside, trust is provided to the authorized end-user by default. However, if an unauthorized end-user was to gain network access, they would have relative free rein to any of the smart devices within the network. As a result, the existence of an external communication pathway could render the host subject's smart device local area network susceptible to a potential security breach of monumental proportion.

In one embodiment, the present invention would utilize a security model that would include a decentralized architecture like blockchain, which allows network participants (i.e., smart medical devices and/or components) to conduct direct interactions with one another without a required intermediary, middleman, or central server. In one embodiment, using this model, peer-to-peer verification is required which for the present invention includes one smart device and/or smart component directly verifying the data transaction with another smart device and/or smart component. In this model of peer-to-peer verification, each smart device verifies and authenticates the identity of its counterpart. Once this peer-to-peer verification has been successfully completed, bidirectional communication can take place, with each smart medical device capable of functioning as a server and/or client. In one embodiment, the data which is being shared is based on the role-based identity of each smart device.

In one embodiment of the present invention, the security architecture utilized is that of Zero Trust, where the program operates on the premise of “Never trust, always verify”. In one embodiment, the general principles of “Zero Trust: are as follows:

### 10. Continuous Monitoring and Validation.

In one embodiment, Zero Trust provides no default trust or access; therefore, all smart devices require continuous identity verification, re-verification, and privileging. In the event that a smart device was incapable of real-time and immediate verification, the program would tender it inoperable and incapable of interacting with another smart device.

### 11. Least Privilege Access.

In one embodiment, least privilege access provides authorized end-users or

devices the minimum amount of data needed to satisfactorily complete the task at hand, analogous to a need-to-know basis for human-to-human interaction and data sharing. This is particularly relevant for the present invention in which smart medical devices will often contain a number of subcomponents, each of which has its own functionality and mission. By the program sharing the minimum amount of data required, the magnitude of a potential security breach and its overall impact on security and safety are minimized.

### 12. Device Access Control.

In one embodiment, in addition to controls on user access, Zero Trust also implements strict controls on device access. The program implements Zero Trust to monitor how different devices are attempting to access the network, ensures each individual device is properly authenticated and authorized, and ensures that each individual device and/or component is secure and has not been compromised or rendered inoperable. This is an important feature of the present invention which relies on smart device and smart component inter-operability, communication, and data sharing.

### 13. Micro-Segmentation.

In one embodiment, micro-segmentation is the practice of breaking up security parameters into small zones, in order to maintain separate access to individual areas of the network. This has extreme practical value to the present invention, given the fact that the host subject local area network is composed of different organ systems, anatomic regions, and individual structures. The ability to compartmentalize smart devices in accordance with host subject anatomy and functionality provides a method for improved security, safety, and performance.

### 14. Multi-Factor Authentication (MFA).

MFA is the requirement for a given end-user or device to undergo multiple data points for authentication and verification, thereby reducing the chance of fraudulently gaining security clearance. In one embodiment, the principles of Zero Trust can be applied to data access and data management, resulting in Zero Trust Data Security, where every request for data access requires dynamic authentication. Data security protocols are predicated and designed based on the attributes of the data, the identity of the end-user or device, and the specific environment and context using Attribute-Based Access Control (ABAC).

### Exemplary Embodiment

In order to illustrate how the present invention and its various features and applications work, take the example of a host subject who is driving alone in a rural location during night time, when he/she experiences acute chest pain, followed by loss of consciousness and crashing of his/her car, which veers off the main road into a nearby culvert, which is out of sight from the main road.

While existing technologies such as smart watches (e.g., 240) may offer some diagnostic assistance in the form of tracking cursory biodata, they are limited in the extent of more detailed diagnosis, and incapable of therapeutic intervention, other than notifying emergency personnel of the host subject physical location. But in this situation, that knowledge is of limited value due to the physically remote location of the host subject and the criticality of his life-threatening condition, requiring immediate intervention if he/she is to survive.

The primary pathology is that of an acute myocardial infarction (MI), resulting in loss of consciousness. As a result, the patient lost control of the car, which in turn crashed, resulting in secondary complications of liver laceration, internal bleeding (i.e., hemorrhage), and shock. The full extent of these pathologies would not be diagnosable by existing superficial health monitoring technologies, like a smart watch.

In contrast, the present invention and its various features/applications provide a methodology for the creation and/or modification of smart medical devices and/or components for both complex pathologic diagnosis and/or treatment, as described above, including (but not limited to):
1. Providing indwelling smart in vivo devices with their attendant attributes.
2. Providing real-time medical data.
3. Ability to make pathologic diagnoses (generalized and targeted).
4. Ability to create and/or modify the dynamic smart Devices and/or components.
5. Providing therapeutic options and decision-making.
6. Providing analysis of treatment response.
7. Providing communication and coordination with external health providers.
8. Providing adaptation of in vivo and ex vivo smart medical devices and/or depots.

In one embodiment, a given host body may have a number of in vivo smart devices and/or components, which routinely are in accordance with pre-existing medical disease, surgical history, pharmacology, and predilection for disease (which can be based upon a number of risk factors including (but not limited to) genetics, occupational history, environmental exposures, and social history).

In this particular use case or exemplary embodiment, the host's in vivo smart medical devices include two coronary artery stents and cardiac pacemaker (based upon pre-existing coronary artery disease and underlying cardiac arrhythmia), Mediport® catheter (venous access for underlying prostate cancer), left hip prosthesis (for treatment old left hip fracture), and circulating nanobots (for disease surveillance). In one embodiment, within these smart devices are a variety of embedded smart subcomponents (e.g., biosensors and miniaturized devices), in both active and inactive states which assist in surveillance, early disease detection, and monitoring of established disease states.

In one embodiment, the program(s) of these in vivo smart devices and components continuously collect real-time biodata; the analysis of which by the program 244 produces a longitudinal record of the patient's comprehensive healthcare status which can be referred to as their “medical profile”. Since the real-time data being collected and analyzed by the program is dynamic in nature, periodic fluctuations in data are routinely noted by the program. This periodic variance in biodata may be a manifestation of normal variability for a given patient (i.e., intra-patient variability), normal variability for a given smart device and/or component (i.e., intra-technology variability), or reflection of early disease. By the program creating context and user-specific medical profiles, the differentiation between normal and abnormal data variability is enhanced.

In the event of a true disease process, real-time biodata and corresponding analysis by the program will demonstrate deviation from the normal medical profile, which can vary in accordance with both the severity and duration of the underlying pathology. In this particular exemplary embodiment, the acute nature of the MI and liver injury resulting in internal hemorrhage and shock will yield profound and rapid changes in biodata, thereby triggering an automated alert by the program 244 by electronic methods (i.e., text, fax, email, etc.) to authorized healthcare providers as well as the internal AI system (e.g., program 234 of device 206, for example) which serves to automate response to real-time events exceeding a pre-determined data threshold.

In one embodiment, the type and diversity of biodata being continuously recorded and analyzed by the program is unique and customizable to both the patient's medical profile as well as the changing biodata itself. In this exemplary embodiment, routine biodata being collected and analyzed by the program will include generalized data reflective of overall well-being (e.g., heart rate, blood pressure, respiratory rate, basic bioassays, temperature, blood chemistries, and cell counts). Additional routine biodata collected by the program and specific to the host's medical profile will be commensurate with pre-existing disease and risk factors including (but not limited to) troponin, D-dimer, myoglobin, C-reactive protein (CRP), and prostate-specific antigen (PSA).

In one embodiment, in the event that statistically significant changes in biodata are detected and verified by the program, a data-specific response is generated by the program based upon the specific type of biodata, its severity, data trending analysis, and available smart device resources (both in vivo and ex vivo). In this particular example, the following abnormalities in real-time biodata are detected by the program:
1. Abnormal physiologic biodata (e.g., elevated heart rate, decreased blood pressure, decreased arterial oxygen).
2. Abnormal bioassays (e.g., elevated troponin, C-reactive protein, D-dimer).
3. Abnormal cell counts (e.g., decreased platelets, decreased red blood cells).

In one embodiment, these changes in biodata are the result of the combined pathologies of acute MI, coronary artery occlusion, liver laceration, and internal hemorrhage. Since some of these abnormal biodata measurements may be caused by multiple disease processes (e.g., decreased blood pressure and increased heart rate), it is important that more specialized biodata be collected and analyzed by the program in order to identify the specific type, location, and severity of the pathology (or pathologies).

In this exemplary embodiment, a number of specialized biodata will be determined by the program to be of benefit including (but not limited to): creatine-kinase MB subtype (CK-MB), myoglobin, cytokine IL-6, heart-type fatty acid binding protein (H-FASP), and B-type natriuretic peptide. In one embodiment, while some of these may be readily available in certain in vivo smart devices like the smart coronary artery stents, others may not be readily accessible and require retrieval from external sources like in vivo or ex vivo device storage depots.

In one embodiment, the quantitative generalized and specialized biodata being collected and analyzed by the program are continuously being supplemented by sensory biodata in various forms, such as electrical, visual, acoustic, and thermal data, etc. In one embodiment, one important source of these sensory data is circulating smart microbots and nanobots (e.g., devices 205, 230), which provide important diagnostic data related to disease (e.g., abnormal coronary artery perfusion, myocardial contractility, active bleeding), as well as anatomic localization. A variety of specialized biosensors (e.g., biosensor 201) and miniaturized devices can facilitate these analyses including (but not limited to): ultrasound transducers, photoacoustic imaging sensors, lasers, micro-optics, optical micro-electromechanically systems [MEMS]. and electrical signal transducers.

In one embodiment, the various ways in which modifications to existing in vivo smart devices has been described above in detail, are directly applicable to the use case. In one embodiment, one such application is the creation of a new in vivo smart device by the detachment of a device segment from its native device, thereby creating an entirely new smart device with functionality specific to its embedded components. In this particular example, once the bleeding site in the liver has been localized by circulating nanobots, a new smart device is created and dispatched to the liver by the program and/or user, for further characterization of the damaged hepatic blood vessel, rate of active bleeding, and size determination of the resulting hemorrhage.

In this exemplary embodiment, one way to accomplish this is through the detachment of a segment of a smart catheter (e.g., catheter 300), followed by the integration of the required miniaturized devices and/or biosensors to perform the required diagnostic and/or therapeutic actions. In one embodiment, to achieve this goal, an individual segment (e.g., segment 302) within the smart catheter detaches itself from the core structure and becomes an independently functioning smart device. Its subcomponents can be derived from a variety of sources including the, for example, Mediport® catheter from which it arose, other in vivo smart devices, an in vivo device depot, or an ex vivo storage depot.

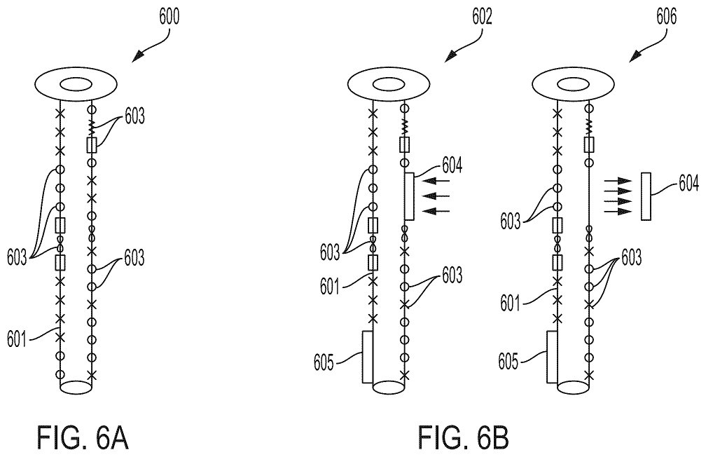

In one alternative embodiment, as shown in FIGS. 6A-B, the new smart device foundation is not derived from a segment of the core catheter (see FIG. 6A where the core catheter 600, includes a number of different biosensors 603 on the body 601 of the catheter), but instead is created as an auxiliary structure 604, 605 (see FIG. 6B) which is connected to but not necessarily a functional component of the smart Mediport® catheter 602 (i.e., redundant smart device design).

In one embodiment, as shown in FIG. 6B, auxiliary structures 604, 605 are attached to the body 601 of the catheter. The auxiliary structures 604, 605 are designed to serve as vestigial components, which when needed, can detach from the parent catheter 602 and create an hoc new smart device 606. In addition, this auxiliary structure 604 can also serve as a storage compartment for additional smart components.

In one embodiment, when the need arises, program AI (or human intelligence) can direct this auxiliary structure 604 to detach itself and become an independent and fully functioning smart device. Once it has been outfitted with the required components (as discussed above), it can navigate to the anatomic site of interest (e.g., right hepatic arterial branch) and begin performing the designated diagnostic and/or therapeutic functions.

In this particular example, the newly created smart catheter 606 may include both diagnostic and therapeutic components 603 such as miniaturized ultrasound transducers for characterization of the bleeding site, biosensors for measuring liver enzymes (e.g., alanine aminotransferase (ALT)) and specialized bioassays (e.g., fatty acid binding protein (L-FABP)). Therapeutic components may include chemoembolization, release of vasoconstrictive pharmacologic agents, cautery of the damaged vessels, and/or surgical ligation of the injured blood vessel(s).

In a similar manner, in one embodiment, one or both the indwelling smart coronary artery stents (not shown) can provide diagnostic and/or therapeutic components for creating a new smart device which can be used in the diagnosis and/or treatment of the left circumflex artery occlusion. In the case of the coronary artery occlusion, therapeutic interventions can include (but are not limited to) balloon angioplasty, release of thrombolytic pharmacologic agents, or physical breakup of the occluding thrombus in combination with trapping of the thrombus fragments.

As previously stated, in one embodiment, deployment of the required smart medical devices and/or components may be directed by program AI 244 or authorized human observers, in accordance with both available or, readily available, resources. In the event that a diagnostic device of importance is not readily available, in one embodiment, remote delivery may be facilitated by drone technology, which can be directed to the specific location of the host subject (e.g., based upon radiofrequency signals transmitted by indwelling smart devices). Once arrived, in one embodiment, these smart devices can be administered by the host if he is conscious or alternatively, the transported smart devices can be administered via robotic technology to the host subject.

In this particular exemplary embodiment, multiple concurrent pathologies are present, which in conventional practice, would likely cause difficulty in diagnosis as well as producing time delays in both diagnosis and treatment. This is because data collection and analysis in conventional practice is performed in series or sequentially. A new diagnostic test is not usually begun until after the results of the first test have been completed, which often results in delayed diagnosis and/or treatment.

In the present invention, however, the program 244 collects and analyzes biodata in parallel, meaning that multiple data are performed and analyzed by the program simultaneously, allowing for faster diagnosis and the ability to diagnose multiple disease processes at once. Since the program of the present invention is continuously collecting real-time biodata from multiple sources, multiple different diseases or pathologies can be both diagnosed and treated at the same time, thereby reducing the time for diagnosis and/or treatment, which in theory can improve clinical outcomes.

In one embodiment, with continuous in parallel collection and analysis by the program, any change in host health stats can be immediately recognized by the program and acted upon by the arsenal of in vivo smart devices and the various ways in which they can be modified and/or transformed in form, content, or functionality.

In one embodiment, in the case of the smart coronary artery stents whose primary function is to diagnose and/or treat coronary artery occlusive disease, the subcomponents already embedded in the core device will readily translate to the new task at hand (i.e., occlusive disease in the left circumflex artery). In the case of the smart catheter being repurposed for diagnosis and/or treatment of acute liver bleeding, it is unlikely that many of the already embedded smart components will translate to the new requirements. As a result, in one embodiment, the requisite components will have to come from a different source, which could take the form of other in vivo smart devices, in vivo storage depot, or ex vivo storage depot.

### SUMMARY

In one embodiment, by having the ability to continuously collect and analyze real-time biodata, the present invention also provides a valuable tool for continuously monitoring and analyzing treatment response. While conventional medical practice routinely assesses treatment response in increments of days, the present invention analyzes treatment response on the order of minutes, which is extremely valuable in highly acute and emergent pathologies. Since small modifications in smart device content and/or functionality may have a significant impact on treatment response, the present invention provides a methodology for monitoring therapeutic impact at both the device and individual component level. If, for example, an individual component was operating in a faulty manner, internal quality control of the smart devices of the present invention can identify the point of failure and lead to replacement of the deficient component.

With the dynamic capability to modify and/or transform smart devices and components on an as-needed basis, the number, availability, and utilization of smart devices and components can undergo rapid change, requiring regular inventory and replenishment, which can also be a function performed by AI. Providing this feedback to technology vendors can also enhance new technology development and innovation, based on the utilization and performance of different smart devices and components.

With continuous real-time data collection and analysis, disease-specific best practice guidelines can be constantly refined and updated and serve as important educational resources for healthcare providers and consumers.

Thus, smart in vivo medical devices and components of the present invention will transform the way medical diagnosis and treatment is performed. The present invention advances the state of in vivo smart devices to a transformative model in which in vivo smart medical devices can now undergo fundamental change in form and functionality, in response to the constantly changing host medical continuum. These structural and functional changes in smart devices and components can be derived in a number of ways, through modification in software and/or hardware. With the ability of the present invention to expand the supply of in vivo smart medical devices and components through the creation of in vivo and ex vivo smart device storage depots, both the practical lifetime of these devices and number of potential transformation options can be enhanced.

In addition, the present invention's use of artificial intelligence (AI) plays an important role in the creation of these pluripotent smart medical devices, for it provides the ability to analyze vast quantities and complexities of real-time data and direct data-driven responses, in accordance with established statistical probabilities and outcomes analysis.

The creation of the technologies of the present invention offers the potential to shift pathologic diagnoses from macroscopic to molecular levels, with the ability to intervene at a far earlier stage in disease, thereby improving clinical outcomes. Thus, in vivo smart devices of the present invention can play an instrumental role in both medical diagnosis and treatment, with the potential of making current and traditional diagnostic and therapeutic techniques and technologies obsolete.

It should be emphasized that the above-described embodiments of the invention are merely possible examples of implementations set forth for a clear understanding of the principles of the invention. Variations and modifications may be made to the above-described embodiments of the invention without departing from the spirit and principles of the invention. All such modifications and variations are intended to be included herein within the scope of the invention and protected by the following claims.

What is claimed is:

1. A medical device which monitors biological data, comprising:
a microscopic medical device configured to be disposed within a human body, said microscopic medical device having a plurality of components which operate in real-time to continuously obtain data from within the human body;
a processor configured to receive said data from said plurality of components and analyze said data;
wherein at least one of said plurality of components is utilized to change a structure, a form, or a content of said microscopic medical device based upon said analysis of said data; and
wherein said microscopic medical device implements a medical intervention within the human body based upon said analysis of said data and said change in structure, form or content of said microscopic medical device.

2. The medical device of claim 1, wherein said change in structure, form or content to said microscopic medical device includes an addition, a removal or a modification of one of said plurality of components.

3. The medical device of claim 2, wherein said microscopic medical device has an articulated structure which allows at least one individual segment of said microscopic medical device to be removed and said microscopic medical device one of deconstructed into separate functioning microscopic medical devices or said individual segment of said microscopic medical device is replaced with another segment introduced externally into said articulated structure.

4. The medical device of claim 2, wherein said microscopic medical device is a biosphere and said plurality of components are a plurality of active, inactive or dormant biosensors which are activated, deactivated, or moved to a different position on the biosphere, to accomplish predetermined activities.

5. The medical device of claim 3, wherein said microscopic medical device comprises one of a microbot or a nanobot, a catheter, a stent, a cardiac pacemaker, a biosphere, an inferior vena cava filter, or a storage device depot.

6. The medical device of claim 5, wherein at least one component of said plurality of components to effect said change in structure, form or content of said microbot or nanobot is introduced from a storage compartment of another microbot or nanobot or from an external storage depot which contains a plurality of components or microscopic medical devices.

7. The medical device of claim 6, wherein a first microbot or nanobot moves to a second microbot or nanobot using its own navigational system, or vice versa, or a third microbot or nanobot delivers said at least one component of said plurality of components from one to the other microbot or nanobot.

8. The medical device of claim 3, wherein said change in structure, form or content to said microscopic medical device changes function of said microscopic medical device and is reversible.

9. The medical device of claim 4, wherein said change in structure, form or content to said microscopic medical device changes function of said microscopic medical device and is reversible.

10. The medical device of claim 3, wherein each said individual segment has its own navigation system.

11. The medical device of claim 3, wherein removal of one of said individual segments or a component of said plurality of components is for repair of said microscopic medical device.

12. The medical device of claim 1, wherein said processor runs a program that utilizes artificial intelligence (AI).

13. The medical device of claim 2, wherein said plurality of components and/or a plurality of microscopic medical devices are constructed or aggregated into a microscopic medical device with different functionality from the individual components and/or devices.

14. The medical device of claim 1, wherein said microscopic medical device is transformed and/or modified using software and/or hardware components.

15. The medical device of claim 1, wherein said processor is remotely activated or deactivated depending upon computer system security requirements.

16. The medical device of claim 5, wherein a plurality of said microscopic medical devices have autonomous function and intercommunication to accomplish said medical intervention.

17. A system which monitors biological data, comprising:
the medical device of claim 1; and
a device depot, which stores one of a plurality of components, a plurality of microscopic or non-microscopic medical devices, or pharmacologic medications, in individual compartments.

18. The system of claim 17, further comprising a plurality of navigational aids which assist said plurality of components or said plurality of microscopic medical devices to navigate and enter said device depot.

19. The system of claim 17, wherein said device depot is internal or external to the human body and said plurality of components or said plurality of microscopic medical devices are injected into the human body from said device depot.

20. A method of monitoring biological data, comprising:
disposing a microscopic medical device within a human body, said microscopic medical device having a plurality of components which operate in real-time to continuously obtain data from within the human body;
receiving data from said plurality of components using a processor, and analyzing said data;
wherein at least one of said plurality of components is utilized to change a structure, a form, or a content of said microscopic medical device based upon said analysis of said data; and
wherein said microscopic medical device implements a medical intervention within the human body based upon said analysis of said data and said change in structure, form or content of said microscopic medical device.
